(19) United States
(12) Patent Application Publication
(10) Pub. No.: US 2022/0033767 A1
(43) Pub. Date: Feb. 3, 2022

(54) ARMORED CELLS

(71) Applicants: Jimin Guo, Albuquerque, NM (US); C. Jeffrey Brinker, Albuquerque, NM (US); Wei Zhu, Albuquerque, NM (US)

(72) Inventors: Jimin Guo, Albuquerque, NM (US); C. Jeffrey Brinker, Albuquerque, NM (US); Wei Zhu, Albuquerque, NM (US)

(21) Appl. No.: 17/277,260

(22) PCT Filed: Sep. 24, 2019
(86) PCT No.: PCT/US2019/052669

§ 371 Date: Mar. 17, 2021

Related U.S. Application Data
(60) Provisional application No. 62/735,585, filed on Sep. 24, 2018.
(60) Provisional application No. 62/808,674, filed on Feb. 21, 2019.

(51) Int. Cl. C12N 5/00; C12N 5/078; C12N 5/0789; A61K 47/69

(52) U.S. Cl. CPC C12N 5/0006; C12N 5/0641; C12N 2500/22; A61K 47/6901; C12N 2500/24; C12N 5/0647

(57) ABSTRACT

A modified vertebrate cell comprising a vertebrate cell encased in reversibly interlinked metal-organic framework (MOF) nanoparticles, and methods of making and using the modified cell, are provided.

### CROSS-REFERENCE TO RELATED APPLICATIONS

This application claims the benefit of the filing date of U.S. application No. 62/808,674, filed on Feb. 21, 2019, and U.S. application No. 62/735,585, filed on Sep. 24, 2018, the disclosures of which are incorporated by reference herein.

### BACKGROUND

Red blood cells (RBCs; also called erythrocytes), are the most abundant cellular constituent of blood and a natural drug delivery system in vertebrates (Yoo et al., 2011). Erythrocytes are special cells in various aspects: they are biconcave shaped, isolated cells without organelles, which exclusively serve as biological carriers. They only exist to distribute and transport various compounds contained within their volume and extended membrane surface. RBCs are responsible for oxygen delivery (O₂) throughout the body. They show prolonged circulation through the vascular system for up to 3 months within mammals and have access to not only components within the blood (Yoo et al., 2011), but also the endothelium and reticuloendothelial system (RES) under physiological conditions. RBCs play a pivotal medical role in various fields including transfusion medicine, or the regulation of the adaptive immune system (e.g. by carrying anti-inflammatory agents or inhibitors of phagocytes). Due to their physiological impact and properties, including biocompatibility, abundance and longevity in circulation, RBCs have been explored over the past decades as carriers of various compounds and nanoparticles and served as an inspirational source of novel functional assemblies and advanced architectures for biomedical applications (Yan et al., 2017; Villa et al., 2016; Villa et al., 2015; Magnani et al., 2014; Wang et al., 2014). Examples range from coupling drugs onto the surface of erythrocytes to improve their delivery and therapeutic effects (Mukthavaram et al., 2014; Godfrin et al., 2012; Pierige et al., 2008), to the attachment of nanoparticles (NPs) onto RBC membrane to alter the circulation behavior of NPs (Brenner et al., 2018; Anselmo et al., 2013; Pan et al., 2018; Anselmo et al., 2015), the encapsulation of RBCs with nanometric films to modulate immune response (Mansouri et al., 2011; Wang et al., 2014; Park et al., 2017; Kim et al., 2016; Park et al., 2016), and the embedding of magnetic NPs in the interior of RBCs to enable magnetic alignment and guidance (Wu et al., Wang & Pumera, 2015).

To date, different strategies have been developed for RBC engineering: 1) Genetic engineering, which causes RBCs to express therapeutic proteins for the treatment of different diseases (Chao & Liu, 2018); 2) Surface grafting, which modifies the RBC membrane by coupling of drugs/targeting agents (Shi et al., 2014); 3) Hypotonic loading, which employs formation of transient pores in plasma membranes in hypotonic solutions to allow the subsequent loading of drugs or NPs in the RBC inner volume (Yan et al., 2017; Villa et al., 2016); 4) Surface hitchhiking, where functional NPs are noncovalently attached to the membrane (Brenner et al., 2018; Anselmo et al., 2013; Pan et al., 2018; Anselmo et al., 2015); 5) Cell-in-shell, which employs encapsulation of RBCs by a nanometric artificial shell, e.g. made of polyelectrolytes, polydopamine, or iron-phenolic networks (Mansouri et al., 2011; Wang et al., 2015; Park et al., 2017; Kim et al., 2016; Park et al., 2016; Park et al., 2014).

Nevertheless, RBCs remain highly sensitive and instable, biological structures that strongly depend on the environment such as the tonicity of chemicals and reaction conditions as well as handling, which strongly hampers current RBC engineering approaches. In order to truly turn RBCs into multifunctional supernanocarrier with externally tunable functions and properties, several limitations needs to be overcome: Current protocols are limited by (i) multiple processing steps and/or long preparation time, (ii) they strongly require the optimization of most material synthesis conditions (such as pH, temperature, precursor concentration) to avoid RBC lysis, and (iii) the lack of versatile capability to endow RBCs with multiple functionalities for biomedical applications.

### SUMMARY

To overcome these limitations, a general strategy was designed to generate stabilized, multifunctional cell-based supercarriers, such as RBC-based supercarriers, that are externally tunable based on a superassembly approach where nanometric metal-organic frameworks (MOFs) act as functional building blocks.

In one embodiment, a modified vertebrate cell, e.g., a viable vertebrate cell having an intact cellular membrane and one or more functions of an unmodified (native) corresponding vertebrate cell, is provided that comprises a coat of reversibly interlinked metal-organic framework (MOF) nanoparticles. In one embodiment, the modified cell comprises a mammalian cell. In one embodiment, the modified cell comprises a human cell. In one embodiment, the modified cell comprises a red blood cell. Red blood cells have a long life in the body, e.g., from about 100-120 days, and can traverse small capillaries, are about 40-50% of the blood volume, and can be easily purified, and so are desirable substrates for preparing armored cells. In one embodiment, the modified cell is a non-adherent cell. In one embodiment, the modified cell comprises a hematopoietic cell. In one embodiment, the modified cell comprises a B cell, T cell or macrophage cell. In one embodiment, the MOF nanoparticles comprise ZIF-8, MIL-100(Fe), UiO-66, UiO-66-NH₂, magnetic iron oxide (Fe₃O₄) NPs@ZIF-8, or mesoporous silica NP@ZIF-8, or a combination thereof. In one embodiment, the nanoparticles have a diameter of about 100 nm to about 500 nm. In one embodiment, the nanoparticles have a diameter of about 100 nm to about 200 nm. In one embodiment, the nanoparticles have a diameter of about 200 nm to about 300 nm. In one embodiment, the nanoparticles have a diameter of about 400 nm to about 500 nm. In one embodiment, the nanoparticles have a diameter of about 450 nm to about 600 nm. In one embodiment, the nanoparticles further comprise covalently attached moieties. In one embodiment, a dye including a fluorescent or luminescent molecule may be attached to the nanoparticles to allow for detection (e.g., imaging or tracking in vivo), e.g., FITC, RITC, Alexa Fluor 488, Alexa Fluor 555, Alexa Fluor 647, or DyLight 800. In one embodiment, the mesopores in the nanoparticles further comprise a moiety, e.g., a diagnostic, e.g., DAR-1, or a therapeutic moiety, e.g., an anti-cancer agent such as doxorubicin. In one embodiment, the mesopores are loaded before the nanoparticles are linked. In one embodiment, the moiety comprises an optically detectable molecule, a diagnostic molecule or a therapeutic molecule. In one embodiment, the diagnostic molecule comprises a contrast agent, e.g., an agent comprising iodine, barium, gadolinium, manganese, or microbubbles, or a magnetic molecule.

In one embodiment, a method of making modified viable vertebrate cells comprising a coat of reversibly interlinked metal-organic framework (MOF) nanoparticles is provided that includes combining a population of vertebrate cells, e.g., intact, functional red blood cells, and a population of MOF nanoparticles under conditions that allow for attachment of the MOF nanoparticles to the surface of the cells, thereby providing a mixture; and adding an amount of a ligand to the mixture under conditions that allow for interconnecting the MOF nanoparticles. In one embodiment, the MOF nanoparticles have a negative charge ranging from −3.0 to −30 mV in 0.2× PBS. In one embodiment, the ligand comprises tannic acid, epigallocatechin gallate, epicatechin gallate myricetin, quercetin, quercetagetin, eupafolin, luteolin, scutellarein, dicaffeoylquinic, theaflavin, heaflavin-3′-gallate (TF1). theaflavin-3, 3′-digallate (TF2) or a combination thereof In one embodiment, the MOF nanoparticles are in an acidic isotonic buffer that alters the zeta potential of the MOF nanoparticles. In one embodiment, the MOF nanoparticles are in a buffer that ranges from pH 5 to pH 7. In one embodiment, the MOF nanoparticles are in a buffer that has a pH less than 7.4.

In one embodiment, the armored cells may be employed to deliver a drug, e.g., doxorubicin, olaparib, altretamine, capecitabine, cyclophosphamide, etoposide, gemcitabine, ifosfamide, irinotecan, melphalan, pemetrexed, topotecan, paclitaxel, or docetaxel, a sensor or any biomolecule cargo (e.g., a protein such as an enzyme, or nucleic acid including but not limited to DNA or RNA) which may be loaded in the pores of MOFs, such as those formed of Fe₃O₄ or Au, or which include MSNs. The MOFs may be further modified to provide for additional functionalities, e.g., so that the armored cells can be detected when they are in a body, can be used as a diagnostic or as a therapeutic delivery vehicle. For instance, if an antibody is attached to the MOF nanoparticles, the armored cell binds to the target of the antibody, or if a prodrug is attached to the MOF nanoparticles, the corresponding drug can be delivered, or a combination thereof which can lead to targeted drug delivery.

In one embodiment, a method of making vertebrate cells comprising a coat of reversibly interlinked metal-organic framework nanoparticles is provided that includes providing a population of viable vertebrate cells, a population of MOF nanoparticles and a ligand; and adding the MOF nanoparticles and ligand, e.g., sequentially, to the cells under conditions that allow for interconnecting the MOF nanoparticles. In one embodiment, the MOF nanoparticles have a negative charge ranging from −3.0 to 30 mV in 0.2× PBS. In one embodiment, the ligand comprises tannic acid, epigallocatechin gallate, epicatechin gallate myricetin, quercetin, quercetagetin, eupafolin, luteolin, scutellarein, dicaffeoylquinic, theaflavin, heaflavin-3′-gallate (TF1), theaflavin-3, 3′-digallate (TF2) or a combination thereof In one embodiment, the MOF nanoparticles are in an acidic isotonic buffer that alters the zeta potential of the MOF nanoparticles. In one embodiment, the buffer ranges from pH 5 to pH 7.

Also provided is a method to disrupt linkages in vertebrate cells comprising a coat of reversibly interlinked metal-organic framework (MOF) nanoparticles, comprising contacting a population of vertebrate cells comprising a coat of reversibly interlinked metal-organic framework nanoparticles with an agent that disrupts the linkages. In one embodiment, the agent comprises a metal chelator. In one embodiment, when armored RBCs squeeze through tiny capillaries in the vasculature (e.g., lung vasculature), the cardiac blood output causes shear force and directs RBC to endothelium contact, thus facilitating transfer of MOF nanoparticles from RBC surface to the pulmonary capillary endothelial cells.

### BRIEF DESCRIPTION OF THE FIGURES

FIGS. 1A-1E. Modular functionalization of RBCs with MOF nanobuilding blocks. A) Schematic representation of Armored RBC formation via immediate, multidentate ligand (tannic acid) assisted super-assembly of MOF nanobuilding blocks on RBC membrane surface. Schematic illustration of the properties of Armored RBCs: in vivo circulation (B), oxygen delivery (C), particle hitchhiking and redistribution (D), and enhanced resistance against external stressors, blood NO sensing, and potential drug delivery (E).

FIGS. 2A-2G. Armored RBC structure characterization. A) Bright field (left) and SEM (right) image of RBCs. Scale bars, 25 μm (left), 2 μm (right). B) TEM and SEM image of UiO-66-NH₂ MOF NPs. Scale bars, 50 nm (left), 500 nm (right). C) SEM image of the armored RBCs-UiO-66-NH₂. Scale bar, 5 μm. D) Confocal fluorescent image of armored RBCs-UiO-66-NH₂ labeled with fluorescein isothiocyanate. Scale bars, 5 μm. E) SEM mapping (Zr, O, and N) of armored RBCs-UiO-66-NH₂. Scale bars, 5 μm. F) Wide XRD patterns of the synthesized armored RBCs-UiO-66-NH₂ and the simulated UiO-66-NH₂ crystals. G) Time-dependent hemolysis of armored RBCs@UiO-66- NH₂ in 1× PBS solution and RBCs in water as control.

FIGS. 3A-3F. Enhanced resistance of Armored RBC to harsh environmental conditions. A) Schematic illustration of the protection of RBCs against external stressors based on MOF NP encapsulation. B) The optical images of human type A, B, and Rh RBCs in their corresponding anti-typing sera. Scale bar, 50 μm. C-E) Hemolytic behavior of native RBCs and Armored RBCs-MIL-100(Fe) as a function of (C) NaCl concentration (i.e. osmotic pressure stimulus), (D) Triton X-100 concentration (i.e. detergent stimulus), and (E) Stöber particle concentration (i.e., NP stimulus). Scale bar is 300 nm. F) The recovery of native RBCs cryopreserved in HES polymer (175 or 215 mg mL⁻¹) PBS dispersions, and Armored RBCs cryopreserved in PBS solution and protected by MIL-100(Fe) NP-based exoskeletons with increasing coating cycles.

FIGS. 4A-4F Assessment of Armored RBC oxygen carrier capability and ex ovo circulation. A) Schematic illustration of the oxygen binding via hemoglobin by RBCs. B) Time-dependent oxygenation curves of native RBCs and Armored RBC-MSN@ZIF-8. Insert images show the generation of bluish glow of native and Armored RBCs after the addition of luminol-perborate mixture. C) UV-Vis spectra of the oxygenated and deoxygenated states of Armored RBC-MSN@ZIF-8 (top), and the related reversible transfer between two states (bottom). The circulation of Armored RBC-MSN@ZIF-8 (D) and native RBCs (E) in the vessel of a chick embryo in the chorioallantoic membrane (CAM model (left), and the related (confocal) fluorescence images (right). Scale bars are 50 μm (left), 5 μm (right). F) The flow of Armored RBC-MSN@ZIF-8 within the CAM capillary bed. Scale bar is 20 μm.

FIGS. 5A-5F. Armored RBCs circulation and biodistribution assessment. A) Whole mice fluorescence images acquired using the IVIS Spectrum at 0.5, 1, 2, 6, 12, and 24 h after intravenous administration of DyLight 800-labeled MSN@ZIF-8 hybrid NPs (top) and the related Armored RBCs (bottom), respectively. Fluorescence images of different organs (liver, spleen, kidney, heart and lung from top to bottom) at 12 and 24 h after intravenous administration of NPs (B) and Armored RBCs (C), respectively. D) Circulation time of both NPs and Armored RBCs (n=3; mean±SD). Insert table is the related elimination half-life. Fluorescence intensity per gram of tissue (E) and relative fluorescence signal per organ (F) at 12 and 24 h after intravenous administration of NPs and Armored RBCs (n=3; mean±SD).

FIGS. 6A-6G. Multifunctional Armored RBCs as tools in nanomedicine. A) Controlled disassembly of the shell leads to a reversible recovery of the RBC normal state for Armored RBC-UiO-66-NH₂ labeled via fluorescein isothiocyanate. The etching time in presence of EDTA amounted to 0, 5 and 15 min. Scale bars, 5 μm. B-C) Armored RBCs serving as a sensor. B) Schematic illustration of the sensing of NO within blood vessels based on the use of the fluorescent probe of DAR-1. C) Calibration curve: fluorescent intensity change versus NO concentration. The red star points out the NO concentration in fresh blood based on the sensing function of DAR-1 loaded Armored RBCs. Insert fluorescent image is DAR-1-loaded Armored RBCs after incubation in NO solution (100 μM) for 5 min. D-E) Armored RBCs as imaging contrast agent. D) Schematic illustration of the manipulation of magnetic Armored RBCs via an external magnetic field (left). Photographs of a dispersion of magnetic Armored RBCs before (left) and after (right) placing a magnet on its side. E) Bright-field microscopy images of magnetically-actuated Armored RBC-Fe₃O₄@ZIF-8 (left) or immobile RBCs (right). Scale bars, 10 μm. F) Fluorescence spectra of multifluorescent Armored RBCs-MSN@ZIF-8 with emission at 428, 515, 648nm. G) Multimodal armored RBCs. Confocal images of multi-fluorescent Armored RBCs based on three different fluorescent MSN@ZIF-8 nanobuilding blocks. Scale bar, 5 μm.

FIG. 7. Zeta potential of MOF NPs at different pH.

FIG. 8. Optical image of native RBCs and Armored RBC-UiO-66-NH₂.

FIG. 9. Bright field image of Armored RBC-UiO-66-NH₂.

FIG. 10. FTIR spectra of UiO66-NH₂ NPs, native RBCs, and Armored RBC-UiO-66-NH₂.

FIG. 11. Bright field image of Armored RBC-MIL-100(Fe).

FIG. 12. UV-Vis spectra of the oxygenated and dexoygenated states of the native RBCs.

FIG. 13. The optical image of chicken embryo.

FIG. 14. TEM image of the NP of MSN@ZIF-8.

FIG. 15. The flow of native RBCs in the capillary. Scale bars, 20 μm.

FIG. 16. Semilog plot of the circulation time of the MSN@ZIF-8 NPs and the related Armored RBCs.

FIG. 17. TEM image of the NPs of Fe₃O₄@ZIF-8.

### DETAILED DESCRIPTION

Bio/artificial hybrid nanosystems based on biological matter and synthetic nanoparticles holds great promises to revolutionize the field of nanomedicine. Herein, armored cells such as ‘Armored Red Blood Cells’ (Armored RBCs), which are native cells super-assembled and protected by a functional exoskeleton of interlinked metal-organic framework nanoparticles (MOF NPs), are described herein. MOFs are periodic and atomically well-defined porous crystalline materials that are typically self-assembled by metal nodes and organic ligands, offering structural diversity, high surface area, tunable porosity, and due to their hybrid nature, the ability to independently functionalize the external and internal surfaces (Furukawa et al., 2013; Denny et al., 2016; Horcajada et al., 2012). This enables the design of MOFs for a spectrum of applications including gas storage and separation, water harvesting, sensing, energy, drug delivery, and acting as nanobuilding blocks for the construction of complex hierarchical nanoarchitectures.

For example, Armored RBCs described herein preserve the original properties of RBCs, and inherit those of MOFs NP, e.g., show enhanced resistance against external stressors. By modifying the physicochemical properties of MOF NPs, Armored RBCs provide diagnostic properties like blood nitric oxide sensing or contrast for multimodal imaging. The synthesis of Armored RBCs is reliable and reversible allowing for stepwise disassembly into distinct building blocks. Its general applicability allows its application to not only any kind of MOF nanoparticles but also to any cell type. The armored cells described herein enlarge the tool box of hybrid nanomedicines to unlock their potential for different fields ranging from biomedical imaging detection and therapy to targeted 3D micropattering in cells and even personalized medicine.

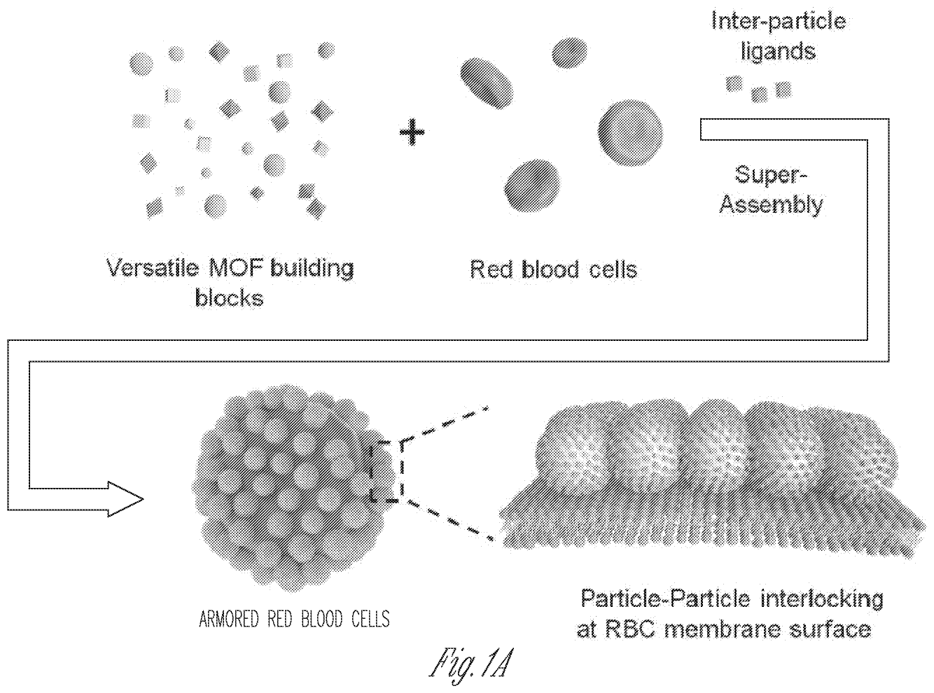

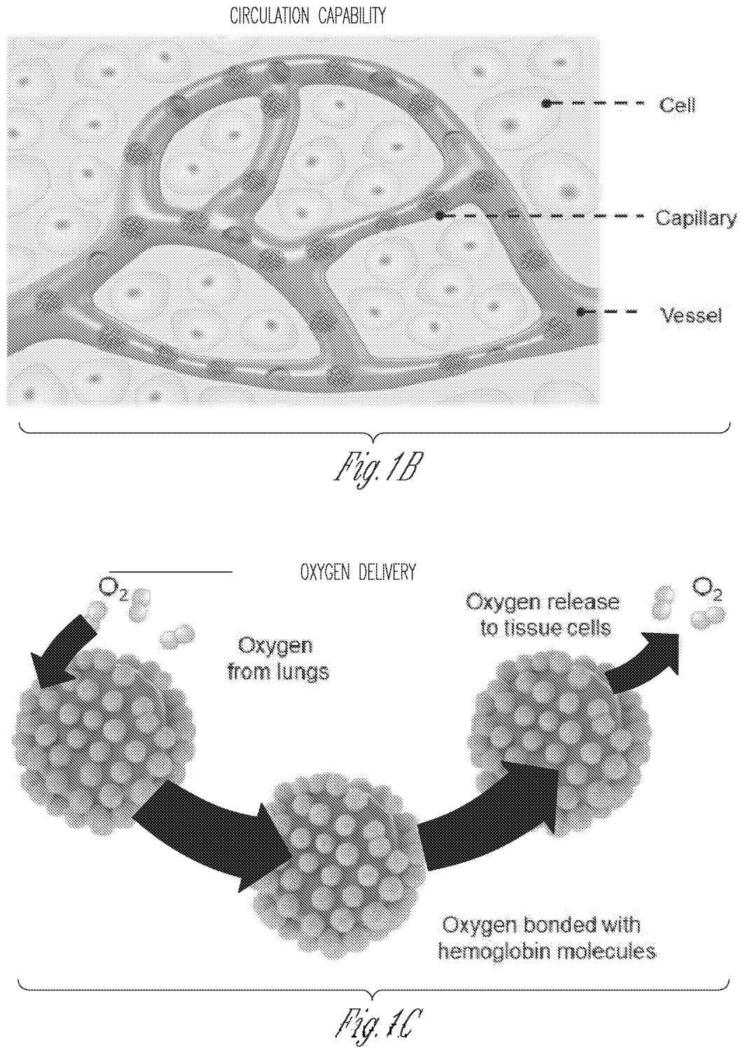

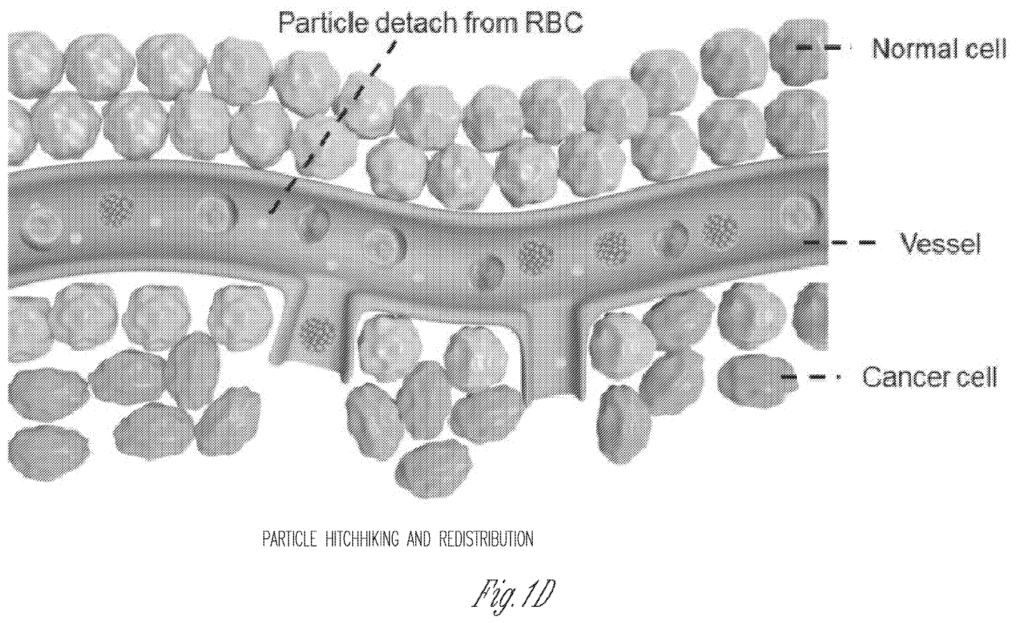

In one embodiment, RBCs are encapsulated, e.g., the surface engineered with functional, modular, MOF nanobuilding block-based exoskeletons (FIG. 1). In one embodiment, RBCs have a diameter of about 7 microns and MOF NOPs have a diameter of about 50 to about 500 nm. Once assembled, the diameter of armored RBCs may be increased to about 105%, 115% or 120% that of native RBCs. Exoskeletons are constructed within seconds through fast MOF NP super-assembly based on strong-multivalent metal-phenolic coordination (Guo et al., 2016; Ejima et al., 2013) and RBC/MOF complexation via multiple hydrogen-bonding interactions at the cellular interface (FIG. 1a). The coating approach is highly biocompatible. It does not introduce, for example, RBC hemolysis nor affect the normal physiology of RBCs in terms of, e.g., oxygen-carrying capability or circulation behavior, as confirmed by the presence a reversible oxygenated and deoxygenated state and long circulation times as determined in chicken embryo and mice models, respectively. Depending on the type of MOF NPs or NP combinations, the physico-chemical properties (e.g., optical, magnetic, and/or sensing properties) of Armored RBCs are highly tunable. The potential chemical diversity of Armored RBCs is enormous, and here various functional MOF NPs including ZIF-8, MIL-100 (Fe), UiO-66-NH₂, magnetic iron oxide (Fe₃O₄) NPs@ZIF-8, and hybrid mesoporous silica NP@MOF (MSNs, dye-labeled MSNs, sensing probe-loaded MSNs@ZIF-8), have been prepared for diverse Armored RBC prototype construction. The newly created Armored RBCs not only show enhanced tolerance against external stressors such as antibody-mediated agglutination, detergent/toxic NPs caused lysis, osmotic stress, and freezing, but also possess RBC abiotic properties including controlled disassembly, multi-fluorescence, magnetism, and blood nitric oxide (NO) sensing, which are foreign to the native RBCs. Taken together, the versatile RBC coating strategy holds great promise to promote the design of MOF/RBC-inspired functional microarchitectures for a wide range of bioapplications.

The invention will be further described by the following non-limiting example.

### EXAMPLE

### Materials and Methods

Reagents. All chemicals and reagents were used as received. Zinc nitrate hexahydrate, 2-methylimidazole, zirconium(IV) chloride, 2-aminoterephthalic acid, dimethylformamide (DMF), iron(III) chloride hexahydrate, trimesic acid, tetraethyl orthosilicate (TEOS), (3-aminopropyl)triethoxysilane (APTES), ammonium hydroxide, ammonium nitrate, hexadecyltrimethylammonium bromide (CTAB), cyclohexane, tannic acid, ethylenediaminetetraacetic acid, fluorescein isothiocyanate (FITC), iron(III) acetylacetonate (Fe(acac)₃), benzyl alcohol, methanol, Ham's F-12K (Kaighn's) medium, Iscove's modified Dulbecco's media (IMDM), formaldehyde solution (36.5-38% in H₂O), and dimethyl sulfoxide (DMSO) were purchased from Sigma-Aldrich. Alexa Fluor™ 647 NHS ester (succinimidyl ester) and DyLight 800 NHS ester were purchased from Thermo Fisher Scientific. Heat-inactivated fetal bovine serum (FBS), 10× phosphate-buffered saline (PBS), 1× trypsin-EDTA solution, and penicillin-streptomycin (PS) were purchased from Gibco (Logan, Utah). Dulbecco's modification of Eagle's medium (DMEM) was obtained from Corning Cellgro (Manassas, Va.). Absolute ethanol was obtained from Pharmco-Aaper (Brookfield, Conn.; 200 proof). Milli-Q water with a resistivity of 18.2 MSΩ cm was obtained from an inline Millipore RiOs/Origin water purification system.

Characterization methods. Scanning electron microscopy (SEM) analyses and energy-dispersive X-ray spectroscopy (EDS) elemental mappings were performed on a Hitachi SU-8010 field-emission scanning electron microscope at 15.0 kV. Transmission electron microscopy (TEM) and high-resolution TEM (HRTEM) imaging were carried out using a Hitachi model H-7650 transmission electron microscope at 200 kV. Wide-angle powder X-ray diffraction (PXRD) patterns were acquired on a Rigaku D/MAX-RB (12 kW) diffractometer with monochromatized Cu Kα radiation (λ=0.15418 nm), operating at 40 KV and 120 mA. The UV-Vis absorption spectra were recorded using a Perkin-Elmer UV/vis Lambda 35 spectrometer. Fluorescence emission measurements were carried out using a fluorescence spectrometer (Perkin-Elmer LS55). Three-color images were acquired using a Zeiss LSM510 META (Carl Zeiss MicroImaging, Inc.; Thornwood, N.Y., USA) operated in channel mode of the LSM510 software.

### Nanoparticles Synthesis

UiO66-NH₂ NPs synthesis. UiO-66-NH₂ NPs were synthesized following previously reported methods (Yoo et al., 2011) with no modification. Briefly, 25.78 mg ZrCl4 (0.11 mmol) and 14.49 mg 2-aminoterephthalic acid (0.08 mmol) were dissolved in 10 mL of DMF solution. Then 1.441 g acetic acid (0.024 M) was added into the above solution. The mixed solution was placed in an oven (120° C.) for 24 h. After cooling down the reaction mixture to room temperature, obtained NPs were subsequently washed with DMF and methanol via centrifugation redispersion cycles. The synthesized UiO-66-NH₂ NPs were stored in EtOH before use.

MIL-100(Fe) NPs synthesis. MIL-100(Fe) NPs was synthesized following previously reported methods with no modification (Yan et al., 2017). Briefly, 2.43 g iron(III) chloride hexahydrate (9.0 mmol) and 0.84 g trimesic acid (4.0 mmol) in 30 ml H₂O were mixed in a Teflon tube, sealed, and placed in the microwave reactor (Microwave, Synthos, Anton Paar). The temperature of the mixed solution was increased from room temperature to 130° C. under solvothermal conditions (P=2.5 bar) within 30 seconds, and then kept at 130° C. for 4 minutes and 30 seconds, and finally cooled down again to room temperature. The synthesized NPs were centrifuged down and then washed twice with EtOH. The dispersed NPs were allowed to sediment overnight. The remaining supernatant of the sedimented suspension was filtrated (filter discs grade: 391, Sartorius Stedim Biotech) three times to finally yield the MIL-100(Fe) NPs. The synthesized MIL-100(Fe) NPs were stored in EtOH before use.

Mesoporous silica NPs (MSN) synthesis. MSN NPs was synthesized following previously reported methods in our group with no modification (Villa et al., 2016). Briefly, 0.29 g of CTAB (0.79 mmol) was dissolved in 150 mL of 0.51 M ammonium hydroxide solution in a 250 mL beaker, sealed with parafilm (Neenah, Wis.), and placed in a mineral oil bath at 50° C. After continuously stirring for 1 h, 3 mL of 0.88 M TEOS solution in EtOH and 1.5 μL APTES were combined and added immediately to the mixed solution. After another 1 h of continuous stirring, the particle solution was stored at 50° C. for another 18 h under static conditions. Next, the solution was passed through a 1.0 μm Acrodisc 25 mm syringe filter (PALL Life Sciences, Ann Arbor, Mich.) followed by a hydrothermal treatment at 70° C. for 24 h. To remove the CTAB, the synthesized MSN NPs were transferred to 75 mM ammonium nitrate solution in ethanol, and placed in an oil bath at 60° C. for 1 h with reflux and stirring. The MSN NPs were then washed in 95% ethanol and transferred to 12 mM HCl ethanolic solution and heated at 60° C. for 2 h with reflux and stirring. Finally, MSN NPs were washed in 95% ethanol, then 99.5% ethanol, and stored in 99.5% ethanol before use.

Fe₃O₄ NPs synthesis. Bare Fe₃O₄ NPs was synthesized following the reported methods with no modification (Villa et al., 2015). Briefly, 0.687 g of Fe(acac)₃ (1.94 mmol) was dissolved in 9 mL of benzyl alcohol. The mixed solution was heated to 170° C. with reflux and stirring at 1500 rpm for 24 h. After the reaction was cooled down to room temperature, 35 mL EtOH was added into the mixed, and then centrifuged at 20000 rpm for 10 min. The supernatant was discarded, and the resulted precipitate was washed with EtOH twice to yield to the Fe₃O₄ NPs. The synthesized Fe₃O₄ NPs were stored in EtOH before use.

MSN@ZIF-8 NPs synthesis. 2.5 mg MSNs were suspended in 2.5 mL water. Next, 250 μL of Zn(NO₃)₂ (0.134 M) and 1 mL of 2-MIM (0.219 M) were subsequently added into the solution. The mixed solution was stirred for 0.5 h, and then centrifuged at 20000 rpm for 10 min. The supernatant was discarded, and the resulted precipitate was washed with EtOH twice to yield to the NPs. The synthesized MSN@ZIF-8 NPs were stored in EtOH before use.

Fe₃O₄@ZIF-8 NP synthesis. 2.5 mg Fe₃O₄ were suspended in 2.5 mL water and then 250 μL of Zn(NO₃)₂ (0.134 M) and 1 mL of 2-MIM (0.219 M) was subsequently added into the solution. The mixed solution was stirred for 0.5 h, and then centrifuged at 20000 rpm for 10 min. The supernatant was discarded, and the resulted precipitate was washed with EtOH twice to yield to the NPs. The synthesized Fe₃O₄@ZIF-8 NPs were stored in EtOH before use.

### RBC Purification

All the animal procedures complied with the guidelines of the University of New Mexico Institutional Animal Care and Use Committee and were conducted following institutional approval (Protocol 11-100652-T-HSC and 17-200658-HSC). Human RBCs were acquired from healthy donors with their informed consent. All blood samples were collected and stored in BD Vacutainer® blood collection tubes (Becton Dickinson, N.J., USA) containing 1.5 mg of EDTA per mL of blood for anticoagulation purposes. The purification of whole blood was carried out using Ficoll® density gradient centrifugation procedure (Magnani & Rossi, 2014).

### Armored RBC construction

Synthesis of Armored RBC-UiO-66-NH₂. 5 million RBCs were suspended in 500 μL of 1× PBS (pH 5) solution containing 400 μg/mL UiO-66-NH₂ NPs . After 10 s vortexing and 30 s of incubation, 500 μL of 40 μg/mL tannic acid in 1× PBS (pH 5) solution was added with 30 s vigorous mixing. The formed Armored RBC-UiO-66-NH₂ was then rinsed with 1× PBS (pH 7.4), and stored in 1× PBS (pH 7.4).

Synthesis of Armored RBC with MIL-100(Fe) NPs coating. 5 million RBCs were suspended in 500 μL 1× PBS (pH 5) solution containing 200 μg/mL MIL-100 NPs. After 10 s vortexing and 20 s of incubation, 500 μL of 32 μg/mL tannic acid in 1× PBS (pH 5) solution were added with 30 s vigorous mixing. The formed Armored RBC-MIL-100(Fe) was then rinsed with 1× PBS (pH 7.4), and stored in 1× PBS (pH 7.4). This process represents a typical procedure for single MIL-100(Fe) NP shell formation and could be repeated one or two times to achieve multi-layered coating.

Synthesis of Armored RBC-MSN@ZIF-8. 5 million RBCs were suspended in 500 μL 1× PBS (pH 5) solution containing 400 μg/mL MSN@ZIF-8 NPs. After 10 s vortexing and 20 s incubation, 500 μL of 32 μg/mL tannic acid in 1× PBS (pH 7.4) solution were added with 30 s vigorous mixing. The formed Armored RBC-MSN@ZIF-8 was then rinsed with 1× PBS (pH 7.4), and stored in 1× PBS (pH 7.4).

Synthesis of Armored RBC-Fe₃O₄@ZIF-8. 5 million RBCs were suspended in 500 μL 1× PBS (pH 5) solution containing 250 μg/mL Fe₃O₄@ZIF-8 NPs. After 10 s vortexing and 20 s incubation, 500 μL of 40 μg/mL tannic acid in 1× PBS (pH 7.4) solution were added with 20 s vigorous mixing. The formed Armored RBC-Fe₃O₄@ZIF-8 was then rinsed with 1× PBS (pH 7.4), and stored in 1× PBS (pH 7.4).

### Hemolysis Assay

Native and Armored RBCs were rinsed with 1× PBS (pH 7.4) solution and then suspended in 1× PBS (pH 7.4) solution at room temperature for 7 days. After centrifugation (300 g, 5 min), the absorbance of hemoglobin in the supernatant was measured by a BioTek microplate reader (Winooski, Vt.) at 540 nm to calculate the hemolysis percentage. Double distilled (D.I.) water and 1× PBS (pH 7.4) solution containing native RBCs were used as positive control (100% hemolysis) and negative control (0% hemolysis), respectively. The hemolysis percentage of each sample was determined using the reported equation (Villa et al., 2016). Percent hemolysis (%)=100×(Sample Abs₅₄₀ₙₘ−Negative control Abs₅₄₀ₙₘ)/(Positive control Abs₅₄₀ₙₘ−Negative control Abs₅₄₀ₙₘ)

### Antibody-Mediated Agglutination

The Antigenic protective capability of Armored RBC was assessed by investigating the attenuation of antibody-mediated agglutination of RBCs. Briefly, 1 million native RBC or Armored RBC-MIL-100(Fe) samples were suspended in 450 μL 1× PBS (pH 7.4) solution, and then 50 μL of anti-type sera that included A, B, and Rh were added. After 15 min, the bright field images were taken by Leica DMI3000 B inverted microscope to evaluate the agglutination.

### Tolerance Against Ion Strength

Native and Armored RBC-MIL-100(Fe) were rinsed with 1× PBS (pH 7.4) solution and then suspended in different concentration of NaCl solution (0 to 0.8%, w/v). After centrifugation (300 g, 5 min), the absorbance of hemoglobin in the supernatant was measured by a BioTek microplate reader (Winooski, Vt.) at 540 nm to calculate the hemolysis percentage.

### Tolerance Against Detergent

Native RBCs and Armored RBC-MIL-100(Fe) were rinsed with 1× PBS (pH 7.4) solution and then incubated in 1× PBS (pH 7.4) with different concentrations of Triton X-100. After centrifugation (300 g, 5 min), the absorbance of hemoglobin in the supernatant was measured by a BioTek microplate reader (Winooski, Vt.) at 540 nm to calculate the hemolysis percentage.

### Tolerance Against Toxic Nanoparticles

Native RBCs and Armored RBC-MIL-100(Fe) were rinsed with 1× PBS (pH 7.4) solution and then incubated in 1× PBS (pH 7.4) with different concentrations of Stöber sphere silica NPs at room temperature for 3 h in continuous rotating state. After centrifugation (300 g, 5 min), the absorbance of hemoglobin in the supernatant was measured by a BioTek microplate reader (Winooski, Vt.) at 540 nm to calculate the hemolysis percentage.

### Cryopreservation and Cell Recovery

The cryopreservation was tested by referring to the reported paper with slight modifications (Wang et al., 2014). Hydroxyethyl starch (HES) were dispersed in 1× PBS (pH 7.4) solution with the concentration of 175.0 and 215.0 mg/mL. 50 million/mL native RBCs and Armored RBC-MIL-100(Fe) were rinsed with 1× PBS (pH 7.4) solution and then suspended in 1× PBS (pH 7.4) solution or HES solution. Each sample was frozen by immersion in liquid nitrogen (−196° C.) for 2 h prior to thawing. Thawing of samples was undertaken by transferring samples to 4° C. in the fridge for a minimum of 2.5 h. Slow thawing process promoted extensively ice recrystallization while ensuring samples were fully thawed. Next, the samples were centrifuged (300 g, 5 min) and the absorbance of hemoglobin in the supernatant was measured by a BioTek microplate reader (Winooski, Vt.) at 540 nm to calculate the cell recovery. Double distilled (D.I.) water and 1× PBS (pH 7.4) solution containing native RBCs were used as the positive and negative controls, respectively. The cell recovery percentage of each sample was determined using the same equation above.

### Chemiluminescence

Luminol-Based chemiluminescence was used to evaluate oxygen carrying capacity of RBCs (Mukthavaram et al., 2014). Briefly, 70 mg sodium perborate, 500 mg sodium carbonate, and 200 mg luminol were added to 5 mL water and dissolved by sonication. The luminol solution was left undisturbed for 5 min in a dark room. For imaging purposes, 1 mL of luminol solution was added to 4 mL samples (20 million native RBC or Armored RBCs) in 1× PBS (pH 7.4) solution. The optical image was taken by a Sony ILCE-5100 Camera (ISO-100 and exposure time of 1/15 s). The chemiluminescence optical image was taken in a dark room by a Sony ILCE-5100 Camera (ISO-6400 and exposure time of 30 s). For luminescence assay, 100 μL of samples in 1× PBS (pH 7.4) solution were added into white 96-well plates at a density of 5 million cells/mL. After that, 20 μL of luminol solution was added to each well and the contents were mixed for 2 min on shaker in the dark. Luminescence was measured using a BioTek microplate reader. The luminescence was expressed as a relative.

### Capability of Reversibly Binding Oxygen

Capability of reversibly binding oxygen was detected by analyzing changes of UV-Vis absorption spectra (300-700 nm) in oxygenated and deoxygenated solutions. For complete deoxygenation, nitrogen gas was in flown into the sample solution to deplete most of the oxygen. After 2 h, sodium dithionite (Na₂S₂O₄) was added, and UV-Vis absorption spectrum was scanned by a BioTek microplate reader. For oxygenation, sample solutions were exposed to atmospheric oxygen for more than 2 h, and UV-Vis absorption spectrum was recorded. This process represents the typical procedure for reversibly binding oxygen capability. Technical replica amount to 3.

### Test of Vascular Flow in Ex Ovo Chick Embryos

The vascular flow characteristics of Armored RBCs were tested using Ex ovo Chick embryo model as described previously (Villa et al., 2016) and was conducted following institutional approval (Protocol 11-100652-T-HSC). Briefly, eggs were acquired from East Mountain Hatchery (Edgewood, N. Mex.) and placed in a GQF 1500 Digital Professional incubator (Savannah, Ga.) for 3 days. Embryos were then removed from shells by cracking into 100 ml polystyrene weigh boats. Ex ovo chick embryos were covered and incubated at 37° C., 100% humidity. 20 million cells/mL of native RBCs and Alexa Fluor 647-labeled-Armored RBC-MSN@ZIF-8 were incubated in 1× PBS (pH 7.4) solution with 10 mg/mL bovine serum albumin (BSA) for 20 min and then rinsed and stored in 1× PBS (pH 7.4) solution. 100 μL of samples in 1× PBS (pH 7.4) solution were injected into secondary or tertiary veins via pulled glass capillary needles. Embryo chorioallantoic membrane (CAM) vasculature was imaged using a customized avian embryo chamber and a Zeiss Axio Examiner upright microscope with heating stage.

### In Vivo Studies on Pharmacokinetics and Biodistribution

All the animal procedures complied with the guidelines of the University of New Mexico Institutional Animal Care and Use Committee and were conducted following institutional approval (Protocol 17-200658-HSC). The experiments were performed on female Albino C57BL/6 mice (6 weeks). To evaluate the circulation half-life of NPs and Armored RBCs, DyLight 800-labeled MSN@ZIF-8 hybrid NPs and the related Armored RBCs were used. Briefly, both samples were incubated in 1× PBS (pH 7.4) solution with 10 mg/mL bovine serum albumin (BSA) for 30 min and then rinsed and stored in 1× PBS (pH 7.4) solution. 150 μL of NPs (1 mg/mL) and the related Armored RBCs (1 mg/mL NPs on Armored RBCs) were injected into the eye of the mice. The blood was collected at 0.5, 1, 2, 6, 12, and 24 h following the injection. Each time point group contained three mice. The collected blood samples were diluted with the same amount of 1× PBS before fluorescence measurement. Particle retention in circulation at these time points was determined by measuring the fluorescence on a BioTek microplate reader (Winooski, Vt.). Pharmacokinetics parameters were calculated to fit a two-compartment model.

To study the biodistribution of the NPs and Armored RBCs in various tissues, 150 μL of NPs (1 mg/mL) and the related Armored RBCs (1 mg/mL NPs on Armored RBCs) were retro-orbital injected to mice. At 12 and 24 h time points following the particle injection, three mice were randomly selected and euthanized. Their liver, spleen, kidneys, heart, lung, and blood were collected. The collected organs were examined with an IVIS fluorescence imaging system (Xenogen, Alameda, Calif.), and the fluorescence intensity of the NPs and Armored RBCs in different organs was further semi-quantified by the IVIS imaging software.

### Armored RBC Shell Controlled Destruction

The Armored RBCs were rinsed with 1× PBS and then suspended in 20 mM EDTA in 1× PBS (pH 7.4) solution for different times (maximum time: 15 min) to allow the controlled destruction of MOF NPs. Then the RBCs were then rinsed with 1× PBS (pH 7.4), and stored in 1× PBS (pH 7.4).

### NO Sensing

Preparation of NO solution followed a reported protocol in NO sensor studies (Godfrin et al., 2012). 10 mM NaOH and 1× PBS (pH 7.4) solutions were pre-bubbled with nitrogen for 2 h to deplete the dissolved oxygen. NO precursor Diethylamine NONOate sodium salt was added to a 10 mM NaOH solution to make the 500 μM stock solution. The stock solution was diluted with 1× PBS (pH 7.4) solutions to generate various concentrations of NO solutions. The NO-containing PBS solutions were set for at least 15 min to allow the NO concentrations to saturate before NO sensor studying. For in vitro NO study, 2.5 million DAR-1-loaded Armored RBCs were suspended in 1 mL of NO-containing PBS solution. For fresh blood NO study, the collected flash blood samples were first diluted with the same amount of 1× PBS, and then incubated with 1 mL solution containing 2.5 million sensing Armored RBCs. After 5 min incubation, the fluorescence emission spectrum was obtained.

### Armored RBC Magnetic Manipulation

The magnetic Armored RBC-Fe₃O₄@ZIF-8 were oriented in the direction of an external magnetic field produced by a neodymium magnet. The bright field images were taken by Leica DMI3000 B inverted microscope to evaluate the magnetic guidance.

### Armored RBC Modular Nanoparticles Super-Assembly

Three different fluorescently labelled MSN@ZIF-8 NPs were used for modular nanoparticles superassembly. For Armored RBC construction, 5 million RBCs were suspended in 500 μL of 400 μg/mL mixed MSN@ZIF-8 nanoparticles (˜1:1:1 ratio) in 1× PBS (pH 5) solution. After 10 s vortex and 20 s incubation, 500 μL of 32 μg/mL tannic acid in 1× PBS (pH 7.4) solution were added with 30 s vigorous mixing. The formed multi-fluorescent Armored RBCs were then rinsed with 1× PBS (pH 7.4), and stored in 1× PBS (pH 7.4).

### Results

### Modular Super-Assembly of Armored RBCs

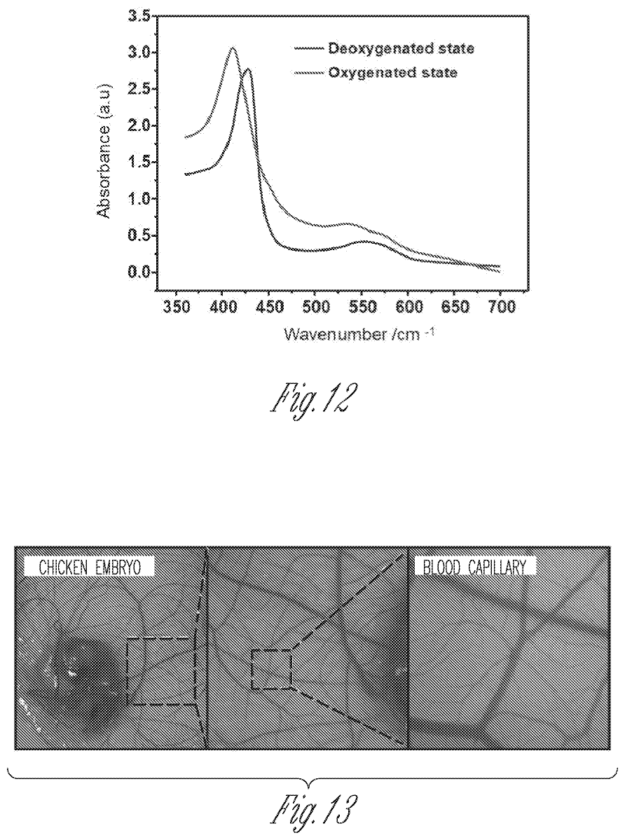

Armored RBCs has a striking advantage for the design of multifunctional, hierarchical nano-assemblies employed for drug delivery and molecular imaging. It can revert to the full range of metal organic framework nanoparticles, as functional, robust and modular building blocks. When constructing a multifunctional, protective shell around single RBCs, these MOF-based NP form a fast exoskeleton based on particle-particle super-assembly and interlocking at the proximal RBC membrane surface. The exoskeleton assembly occurs in two steps: at first, MOF NPs attach and concentrate onto the native RBC surface. Since RBC membranes are rich in carbohydrates and proteins, they have a highly negatively charged surface (Bondar et al., 2012). Due to the frangibility and sensitivity of RBCs, a strong interaction between NPs and RBC membranes always causes RBC rupture and hemolysis. MOF NP surfaces comprise well-defined, long periodic arrangements of metal nodes and organic ligands, which allow precise tuning of the coordination and interactions with organic moieties on the RBC membrane surface. Based on zeta potential (ζ) measurements, MOF NPs used within this study (UiO-66-NH₂, MIL-100(Fe), and ZIF-8) have a negative charge ranging from −3.0 to −29.1 mV (FIG. 12), which should avoid strong electrostatic interactions between the MOF NPs and the negatively charged RBC surfaces (zeta=˜30 mV) thereby suppressing hemolysis; however, the electrostatic repulsion may limit MOF accumulation and attachment. To balance both contributions, an isotonic buffer (phosphate-buffered saline (PBS) at pH 5.0) was selected in which the zeta potential of MOF NPs was greatly decreased and no hemolysis of RBCs occurred. Under these conditions, hydrogen bonding interactions between the organic ligands from MOF NPs and carbohydrates and proteins from RBCs drive MOF accumulation and attachment. The second step of exoskeleton assembly of Armored RBCs involves interlocking of MOF NPs, that are already attached to the RBCs surface and additional exogenous MOFs via an interparticle ligand. Tannic acid was employed as the interparticle ligand and it was added sequentially to the MOF NPs/RBCs mixed solution after short time incubation (˜30 s). Tannic acid is frequently used as an organic building block (Rahim et al., 2018) in MOFs, due to its well-known biodegradability and strong-multivalent coordination binding to various metal ions. Coupled with the additional strong metal-phenolic interactions, the colloidal MOF NP-based exoskeleton in armored RBCs can be created rapidly, in seconds.

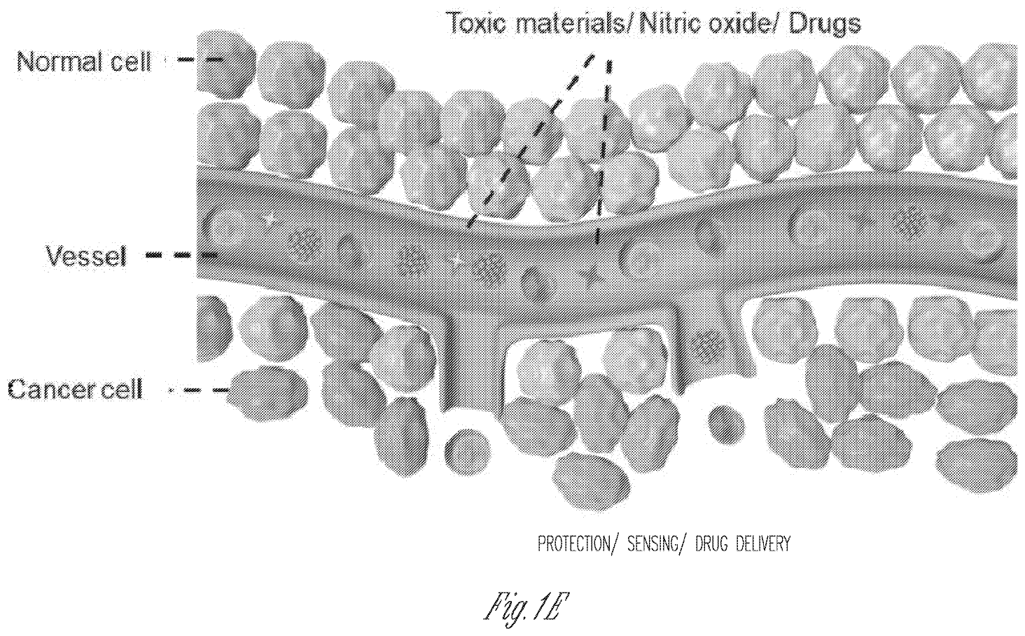

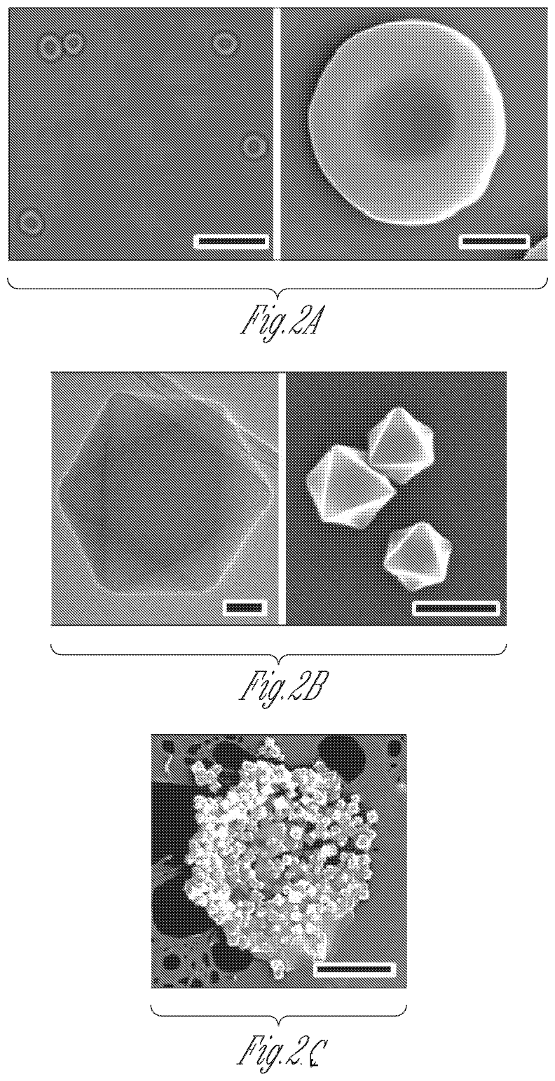

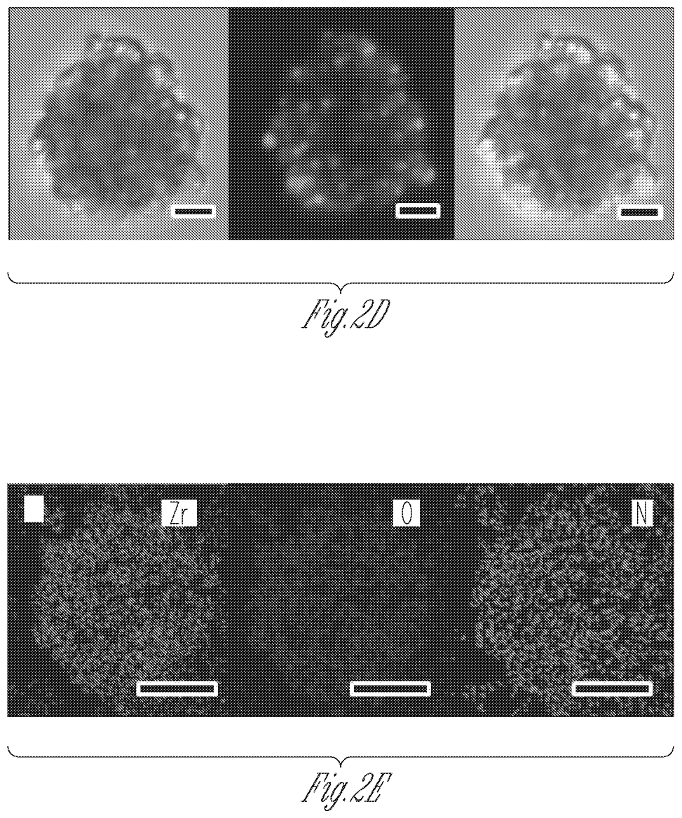

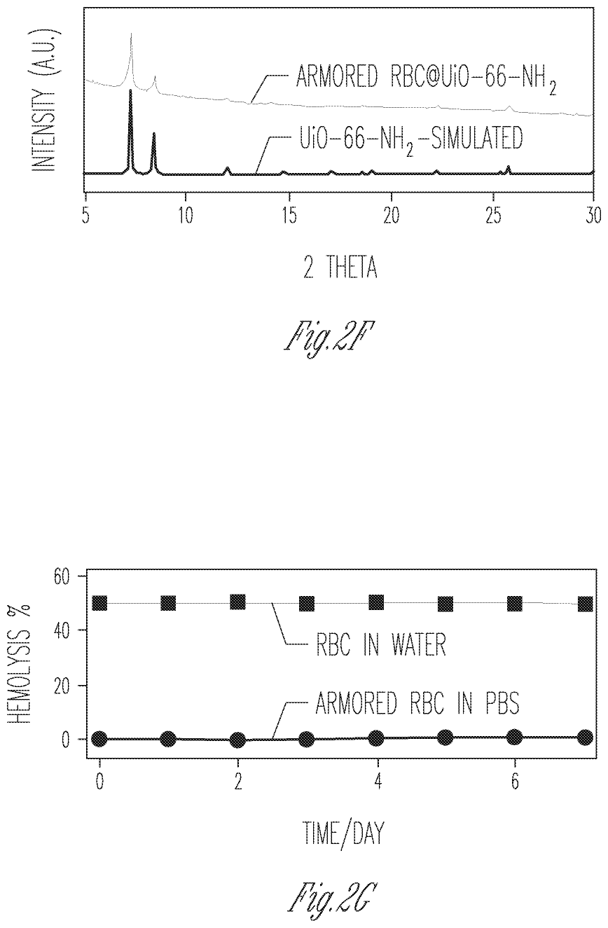

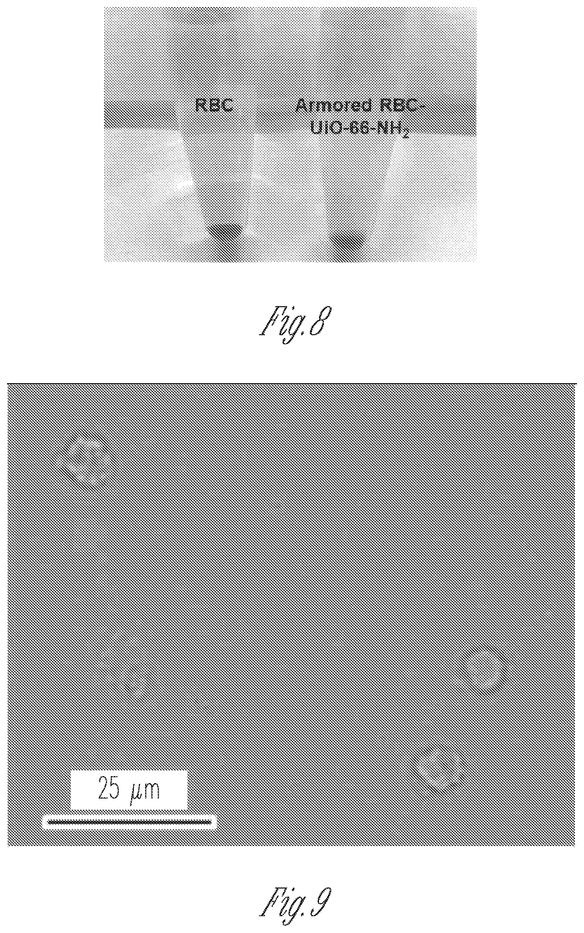

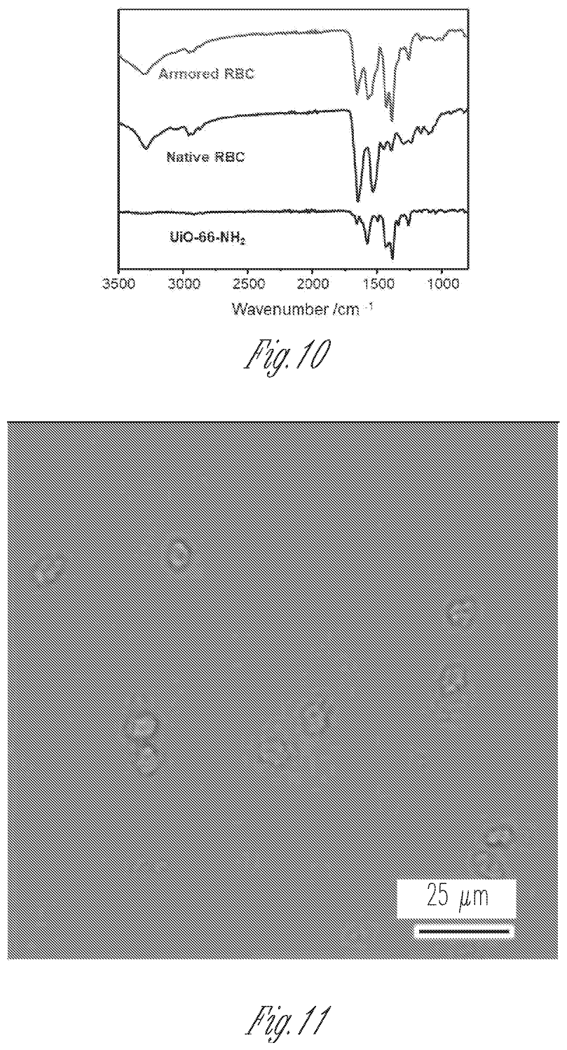

As a demonstration of the armored cell concept, individual purified RBCs (FIG. 2a) encapsulated within UiO-66-NH₂ MOF NP-based exoskeletons (termed Armored RBC-UiO-66-NH₂) were constructed via the sequential addition of a colloidal UiO-66-NH₂ NP solution and tannic acid to RBC suspensions. Colloidal UiO-66-NH₂ MOF NPs with diameter of ˜440 nm were synthesized according to the reported solvothermal methods (Cavka et al., 2008). Transmission and scanning electron microscopy (TEM, SEM) (FIG. 2b) and wide-angle X-ray diffraction (XRD) (FIG. 2f) confirmed their crystalline structures with well-defined octahedral shape. The formation of UiO-66-NH₂-based exoskeletons around RBCs is clearly visible not only by SEM (FIG. 2c) but can be even observed by the naked eye (FIG. 8) as a color change from bright red to light red. The roughened surface (FIG. 2c), which is due to a dense packing of NPs at the RBC surface, is further visible in bright field images (FIG. 9). Fourier-transform infrared spectroscopy performed on Armored RBC-UiO-66-NH₂ (FIG. 10) confirmed the presence of UiO-66-NH₂ MOF NPs, as evidenced by the characteristic peaks at 1570 and 1256 cm⁻¹ assigned to the —CO₂ asymmetrical stretching and vibration of C—N, respectively, in the aminocarboxylate groups of UiO-66-NH₂. Analyzing nearly fifty Armored RBCs on bright field optical (FIG. 9) and SEM images (FIG. 2c) supported the fact that all erythrocytes were encapsulated within homogeneous conformal exoskeletons. This could be further confirmed by confocal scanning laser microscopy of fluorescein isothiocyanate-labeled UiO-66-NH₂ NP-based exoskeletons. A uniform and homogeneous UiO-66-NH₂-NP layer that encapsulates the erythroid cells can be observed (FIG. 2d). Moreover, energy-dispersive X-ray (EDX) spectroscopy for mapping of zirconium, oxygen, and nitrogen atoms (FIG. 2e) along with wide-angle XRD (FIG. 2f) further confirmed, that the structural and chemical integrity of UiO-66-NH₂-NPs are preserved within the exoskeleton. Importantly, the fast coating process did not cause the hemolysis of RBCs even after 7 days storage (FIG. 2g), indicating that the formed exoskeleton had no toxic effects on RBCs. To demonstrate the generality of the Armored RBC approach, i.e., the protective and functional encapsulation of native RBCs by diverse types of MOF NPs via spontaneous super-assembly, MIL-100(Fe), magnetic Fe₃O₄ NPs@ZIF-8, and hybrid MSNs@ZIF-8 in different shapes, sizes and functionalities were tested as building blocks for the design of various Armored RBCs. The results clearly demonstrate that the Armored RBC approach is a powerful and universal strategy to create multifunctional, cellular super-assemblies, as it is a fast, easy, and biocompatible functionalization process of cellular membranes.

### Armored RBCs Show Enhanced Resistance Against External Stressors

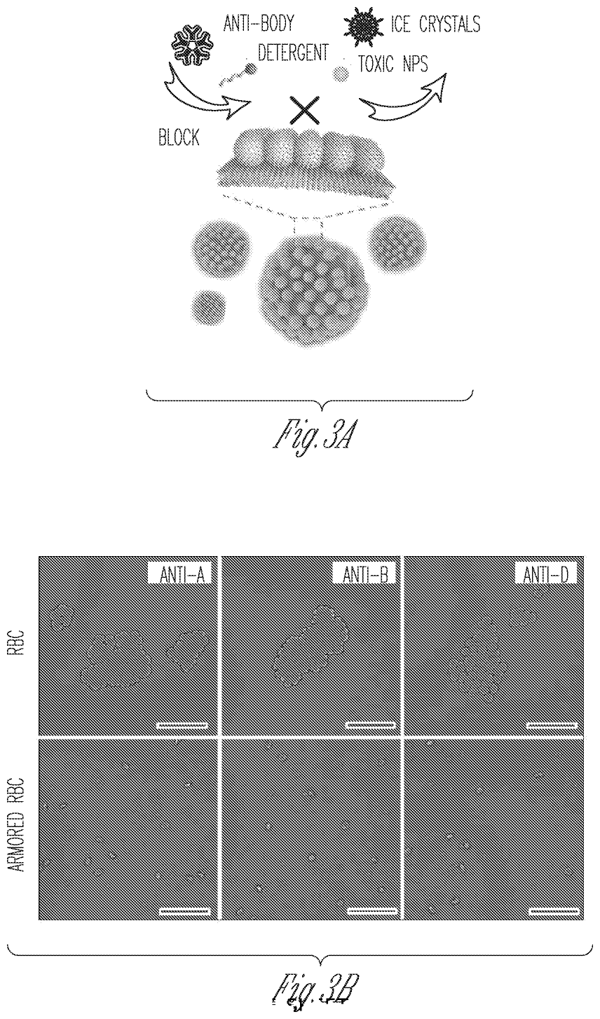

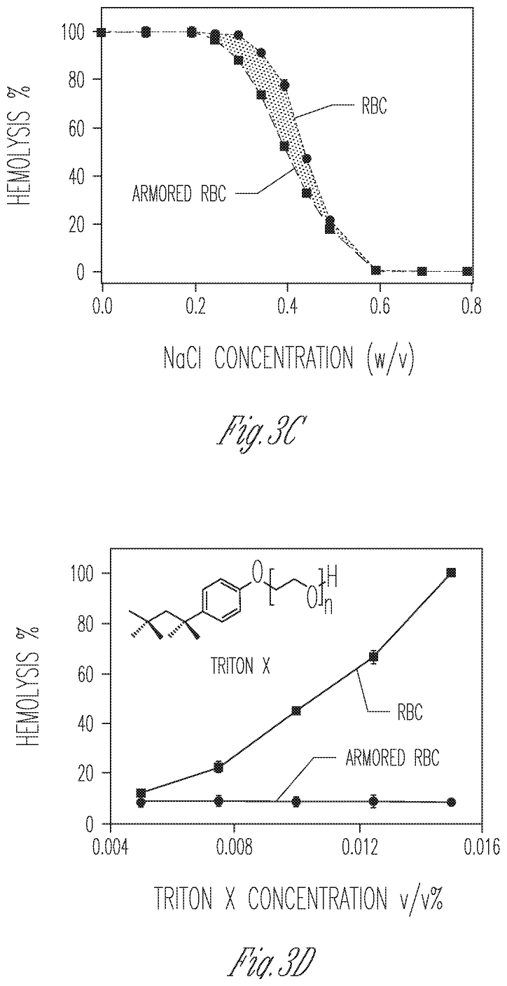

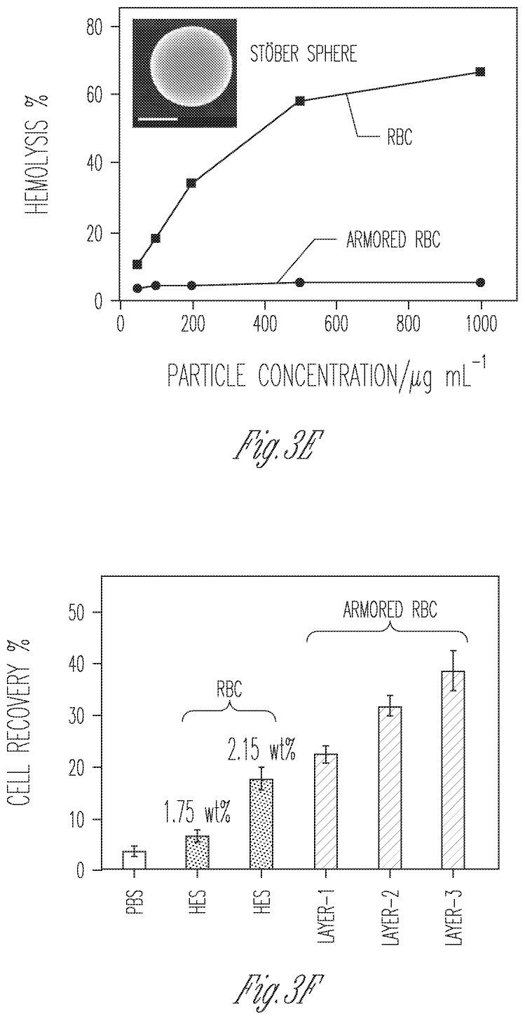

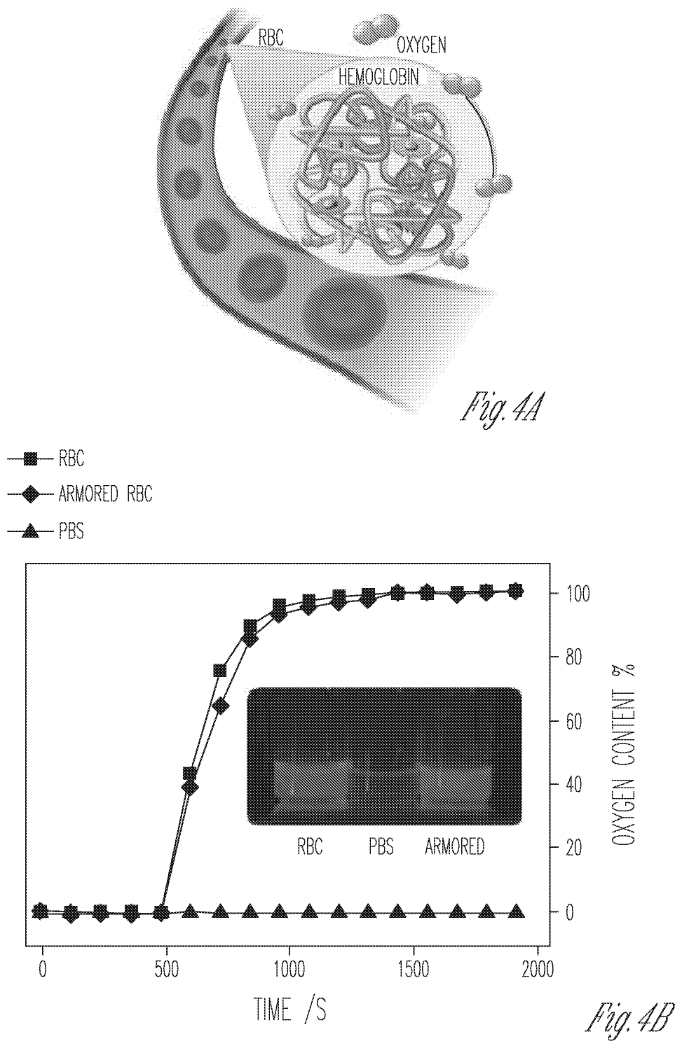

The first impressive property of Armored RBCs is their enhanced cytoprotection against external stressors. To benchmark the protective effect, we exposed armored RBC-MIL-100(Fe) to various harsh environmental conditions including antibody-mediated agglutination, osmotic pressure, detergents, toxic NPs, and freezing conditions (FIG. 3a). The bright field image in FIG. 12 confirmed the successful assembly of a homogeneous MIL-100(Fe)-NP-based exoskeleton. At first, antibody-mediated agglutination assays were performed (FIG. 3b) to assess the blood group antigen immunogenicity of Armored RBCs. This is a factor in RBC alloimmunization (Mansouri et al., 2011), since a cross-type agglutination reaction caused by blood type mismatch during blood transfusion may lead to potentially fatal massive immune hemolysis and even patient death. As shown in FIG. 3b (top row), native RBCs of type B rapidly and severely agglutinated in presence of small amounts of anti-A serum. Identical results were obtained with type A and type RhD-RBCs in their corresponding anti-type anti-sera; on the contrary, no antibody-mediated aggregation was observed in Armored RBCs even with the extension of exposure time (FIG. 3b—bottom row). Armored RBCs possess a highly effective immune-protective exoskeleton that shields the immune-response provoking epitopes on RBC surfaces against agglutination. This protection strategy may allow the survival of foreign RBCs during blood transfusion and thus can act as potential universal RBCs that overcome the blood type mismatch problem. Secondly, the tolerance of Armored RBCs to osmotic pressure was tested, and the percentage of cells undergoing hemolysis measured. At first glance, the osmotic fragility curves of both native RBCs and Armored RBCs showed similar rupture profiles (FIG. 3c). Cellular fractures of native and Armored RBCs were initiated at a concentration of NaCl of 0.60% (w/v), but native RBCs burst completely at a concentration of 0.30%, while the burst concentration of Armored RBCs was shifted to 0.20%. The enhanced tolerance to osmotic pressure can be attributed to two reasons. On one hand, the enhanced membrane reinforcement provided by the MOF exoskeleton offers a physical restriction that delays the swelling process. On the other hand, the potential adsorption of ions in MOF pores may delay the ion transfer from external medium to RBC intracellular fluids (Horcajada et al., 2007). Both effects could lead to a reduced and delayed RBC lysis under hypotonic conditions. Next, the hemolytic protection of Armored RBCs exposed to the nonionic detergent Triton X-100 and so-called ‘Stöber’ amorphous silica NPs was tested, since both species are known to easily cause RBC lysis. As shown in FIG. 3d-e, a slight change in Triton X-100 concentration or particle concentration greater than 500 μg mL⁻¹ already led to massive lysis of RBCs, whereas Armored RBCs exhibited negligible hemolysis under both conditions. This cytotoxic agent resistance is attributed to a physical barrier effect provided by the armored shell. Finally, the resistance of Armored RBCs to freezing conditions in comparison to native RBCs was determined. Ice recrystallization is the major challenge during cryopreservation of RBCs (Biggs et al., 2017). The formation of ice crystals not only incites serious mechanical damages to the delicate RBCs but also creates increased osmotic pressure across the cell membrane, leading to rupture of RBCs. In order to provide extensive ice recrystallization and maximize cell stress during the cryopreservation test, both native RBC and Armored RBC samples were rapidly frozen in liquid nitrogen (−196° C.) for 2 h and then slowly thawed at 4° C. over several hours. As shown in FIG. 3f, the native RBC recovery in PBS solution is very low (<5%). However, protected by the armored shell, the cell recovery increased to ˜25%. With further increase of coating cycles to get a thicker shell, the cell recovery of RBCs can be increased up to 40% without the addition of any toxic solvents. This cell recovery is superior to that obtained via commonly used hydroxyethyl starch polymers (HES) at concentration of 1.75 wt % and 2.15 wt %, highlighting the excellent protection conferred by the MOF NP-based exoskeletons.

### Assessment of Armored RBCs with Respect to their Oxygen Carrier Capability, Ex Ovo and In Vivo Circulation and Biodistribution

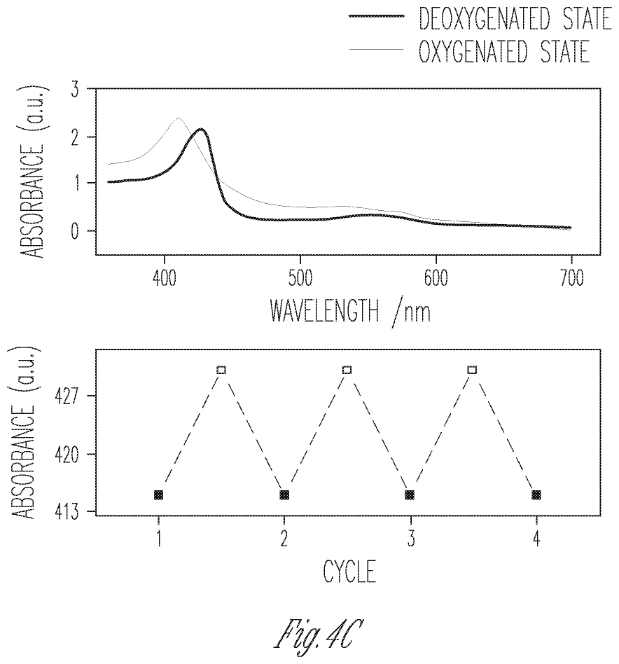

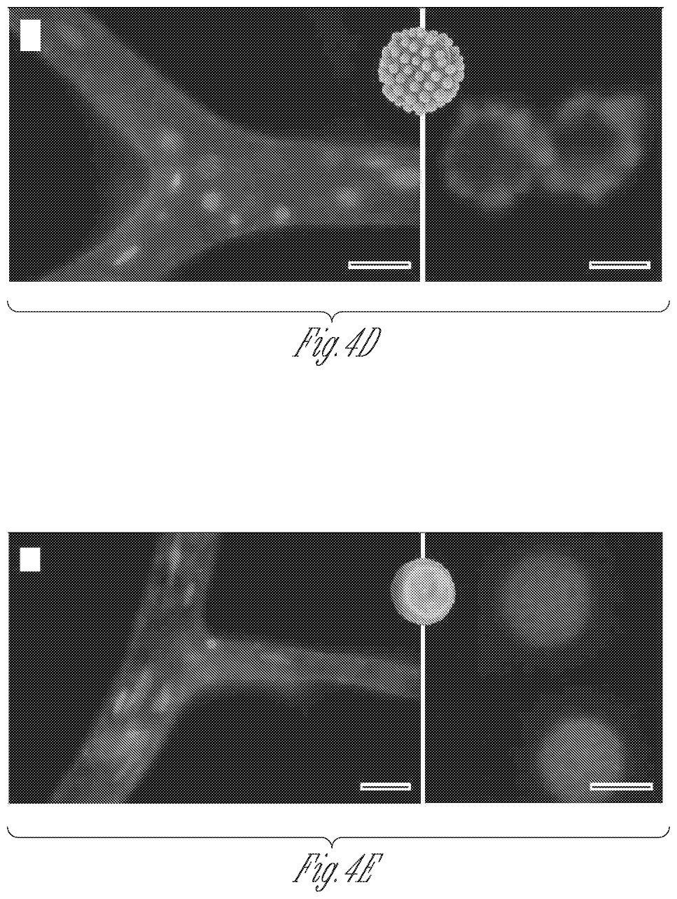

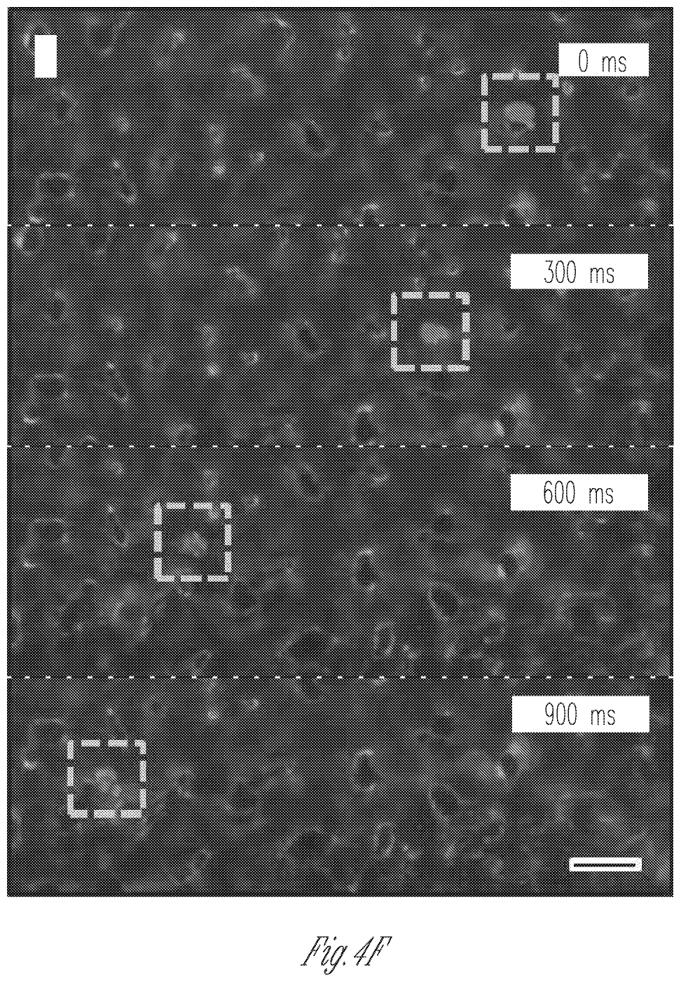

An important feature of RBCs is their oxygen carrier capability and long-circulation times in blood. To facilitate the in vivo bioapplication of the designed Armored RBCs, Armored RBCs must exhibit the very same behaviors as native RBCs. At first, luminol-based chemiluminescence was employed to reveal the presence of hemoglobin in Armored RBCs (FIG. 4a). When hemoglobin and luminol-perborate mixture come in contact, the iron in the hemoglobin accelerates the reaction of luminol with the peroxide generated from perborate, which results in a bluish glow (Inset of FIG. 4b). After addition of luminol-perborate mixture in solution, both native RBCs and Armored RBCs-MSN@ZIF-8 become chemiluminescent after 10 min. This clearly shows that the armored shell does not inhibit the iron catalytic property of hemoglobin inside RBCs. The inherent porosity of the armored shell provides full accessibility of small molecules such as luminol and peroxide to the RBC and permits access to and crossing of the RBC membrane. To investigate the oxygen carrier capability, UV-Vis spectroscopy was used to reveal the reversible shift of maximum absorption peaks of RBCs in oxygenated and deoxygenated states (FIG. 4c and FIG. 12). The characteristic absorption peak of native RBCs and Armored RBCs in an oxygenated state appeared at 415 nm (FIG. 4c and FIG. 12). After bubbling nitrogen for 2 h and adding the reducing agent sodium dithionite (Na₂S₂O₄), the absorption peak red-shifts to 430 nm, confirming the switch to the deoxygenated state for both native RBCs and Armored RBCs. The deoxygenated state can carry oxygen again after exposure to atmospheric oxygen. The process of binding and releasing oxygen was repeatable (FIG. 4c), demonstrating that the oxygen carrier capability of Armored RBCs was preserved. By comparing the oxygenation rate of both deoxygenated samples, as shown in FIG. 4b, the time-dependent oxygenation curves of native and Armored RBCs showed the similar behaviors, while a ˜60 s delay was found for Armored RBCs. This may be explained by the presence of the porous MOF shell that increases the tortuosity of the oxygen diffusion pathway, and thus causes oxygen molecules to spend more time trapped within MOF pores.

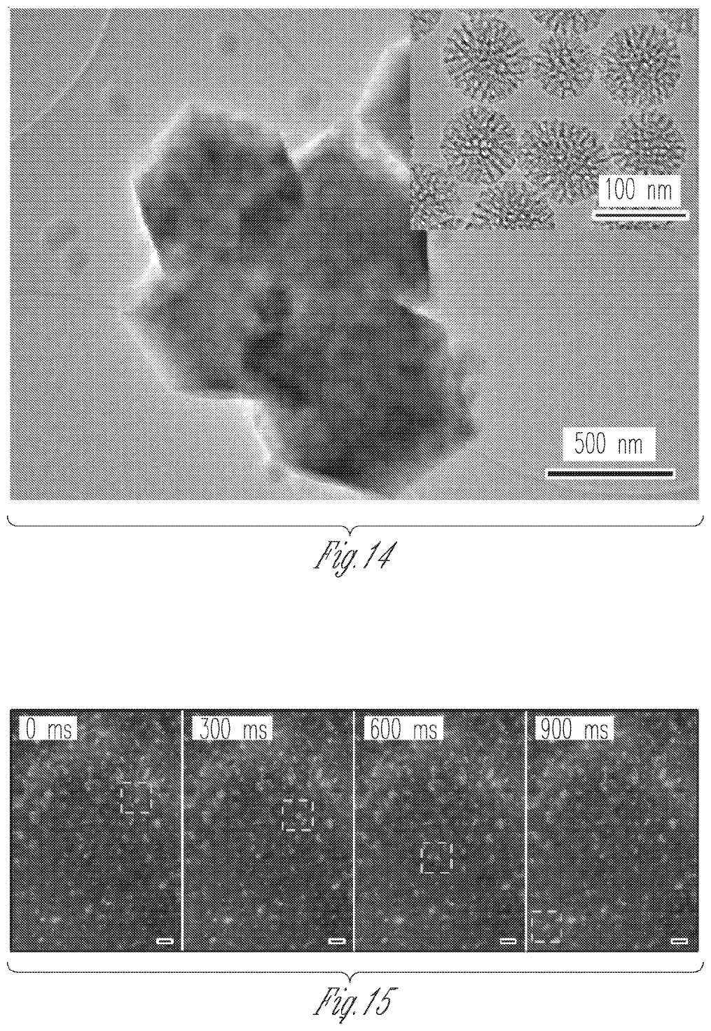

RBCs are well known to easily traverse the microvasculature with dimensions that are smaller than their size and display long circulation times in vivo. To investigate the circulation behavior of Armored RBCs, real-time fluorescence wide-field imaging was carried out on a chick embryo ex ovo (chorioallantoic CAM model; FIG. 13), that provides easy and direct optical access for intravital imaging of the flow of RBCs in blood vessels. Alexa Fluor 647-labeled MSN@ZIF-8 hybrid NPs with diameter of ˜80 nm were used as building blocks for Armored RBC construction (FIG. 14). A coherent, conformal NP layer encapsulating RBCs, clearly observed in FIG. 4d, confirmed the successful formation of Armored RBCs. The blood vessels are labeled with fluorescein labeled lens culinaris agglutinin (LCA) for direct visualization. As shown in FIG. 4d,f, compared to the flow of the native RBCs in the blood vessels and capillaries (FIG. 4e and FIG. 15), the flow of the Armored RBCs was not affected, even after circulating for 30 min, supporting normal circulation properties, despite the Armored RBCs shape exhibiting a certain degree of deformation compared to native RBCs.

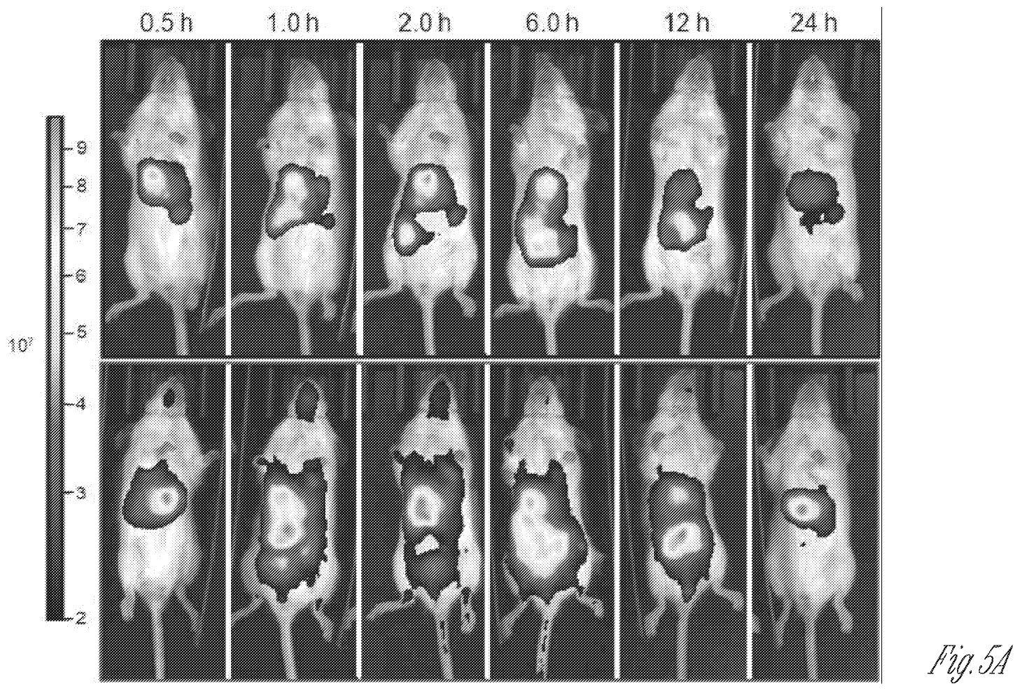

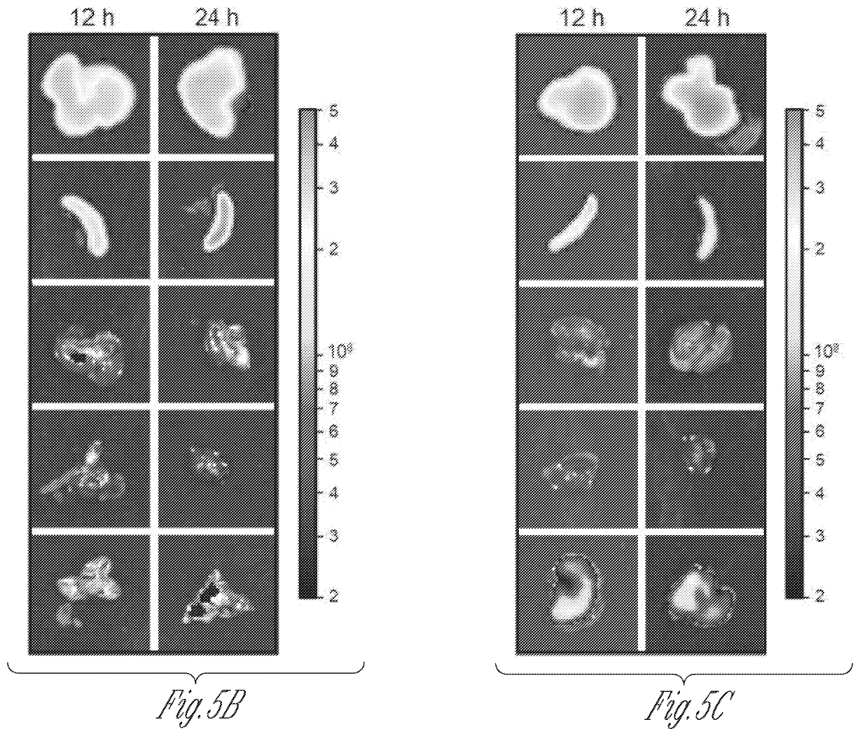

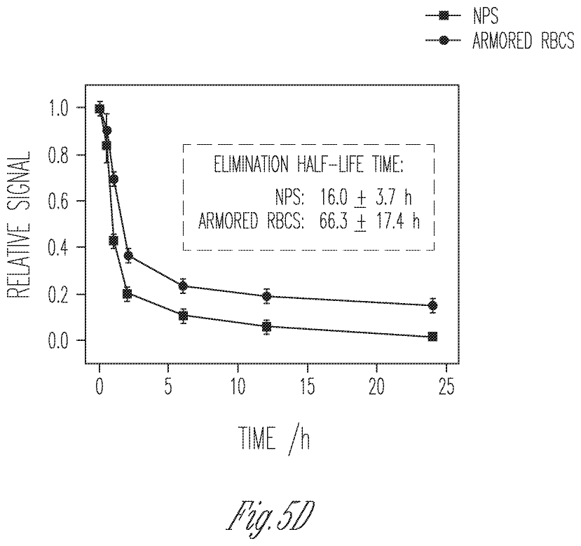

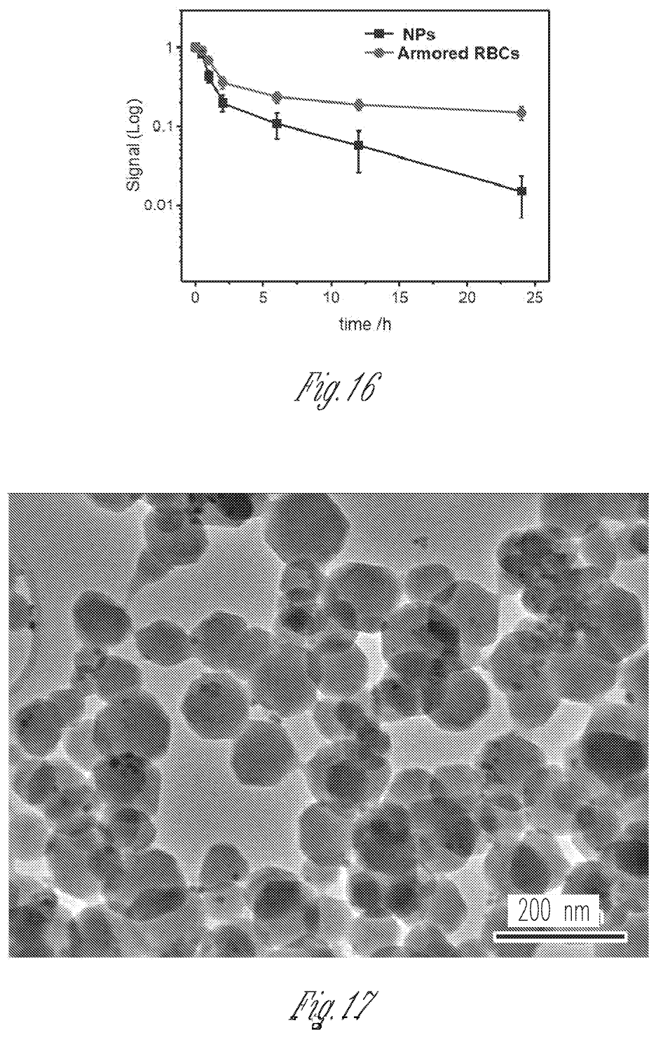

To further characterize circulation properties of the Armored RBCs in vivo, albino C57BL/6 mice were used to examine their pharmacokinetic and biodistribution behaviors. Mice were injected with control DyLight 800-labeled MSN@ZIF-8 hybrid NPs and the corresponding MSN@ZIF-8 Armored RBCs by retro-orbital injection, respectively, at a dose of 150 μg NPs/mouse. Syngeneic RBCs were used to create Armored RBCs, negating blood cell type complications. To study the circulation half-life, at various time points following the injection (FIG. 5a), blood was collected from the eye socket of the mice to evaluate the concentrations of circulating control NPs or Armored RBCs. At 12 h and 24 h post injection, the Armored RBCs exhibited 19% and 15% overall retention in mice blood, respectively, as compared to the 6% and 2% exhibited by control NPs (FIG. 5d). The semilog plot of retention-circulation time (FIG. 16) illustrates a bi-exponential decrease in particle concentration over time, indicating that both NP and Armored RBC circulation followed a two-compartment pharmacokinetic model. After fitting to the two-compartment pharmacokinetic model, numerical analysis (FIG. 5d) indicated that the elimination half-life of NPs and Armored RBCs was 16.0±3.7 h and 66.3±17.4 h, respectively. Armored RBCs displayed a remarkably enhanced retention in blood circulation in comparison with control NPs. The present finding is well correlated with the reports that the anchoring of particles onto the RBC surface could prolong the intravascular particle circulation, where the flexibility, circulation, and vascular mobility of RBCs could help the adhered nanoparticles to escape rapid reticuloendothelial system (RES) clearance (Brenner et al., 2018; Anselmo et al., 2013; Pan et al., 2018; Anselmo et al., 2015).

Furthermore, to analyze the related biodistribution, at 12 h and 24 h post injection, mice were euthanized and their liver, spleen, kidneys, heart, lungs, and blood were harvested for fluorescence analysis (FIG. 5b). The majority of fluorescence signal was found in the two primary filtering organs, the liver and spleen after 12 h post injection, supporting removal by the RES. However, the fluorescence intensity from the Armored RBCs in the spleen decreased 24 h post injection and simultaneously increased in the lungs, suggesting detachment of MOFs from the RBC surface and their return into circulation (FIG. 5a). Notably, 24 h after injection, the Armored RBCs exhibited a 10-fold higher blood persistence and 3.5-fold higher accumulation in lungs compared to the control NPs (FIG. 5e-f). It was postulated that NPs from Armored RBCs accumulate in the lungs following release caused by squeezing through tiny capillaries in the lung vasculature (Brenner et al., 2018; Anselmo et al., 2013; Pan et al., 2018; Anselmo et al., 2015). The high cardiac blood output can cause shear forces that could facilitate the transfer of the NPs from RBC surface to pulmonary capillary endothelial cells.

In summary, Armored RBCs exhibit enhanced in vivo circulation/residence times compared to many other classes of nanoparticles (Brenner et al., 2018; Anselmo et al., 2013; Pan et al., 2018; Anselmo et al., 2015) and could serve as sources of MOFs (or conceivably other nanoparticles) that could be released and targeted to various organs over time. This could extend the in vivo applications of MOFs and enable targeting and delivery to difficult-to-reach sites in the body.

### Multifunctional Armored RBCs Construction

Given the chemical diversities of MOF nanobuilding blocks, the armored RBC concept can be generally extended based on a plethora of MOF types and combinations. It is basically unlimited in generating diverse functionalities and hence serves as a promising technology to satisfy the growing need of multifunctional nanoparticles in biomedical applications, which we demonstrate in the following section. Depending on the MOF building block, armored MOF shells can (i) not only be created but also be biocompatibly disassembled if necessary. Depending on the underlying MOF NPs, armored RBCs offer (ii) unique physiochemical properties such as optical, magnetic and sensing properties. But most importantly: (iii) the armored RBC concept profits enormously from multiplexing: MOF NP associated properties can be linearly combined within the Armored RBC shell during a mixed super-assembly synthesis.

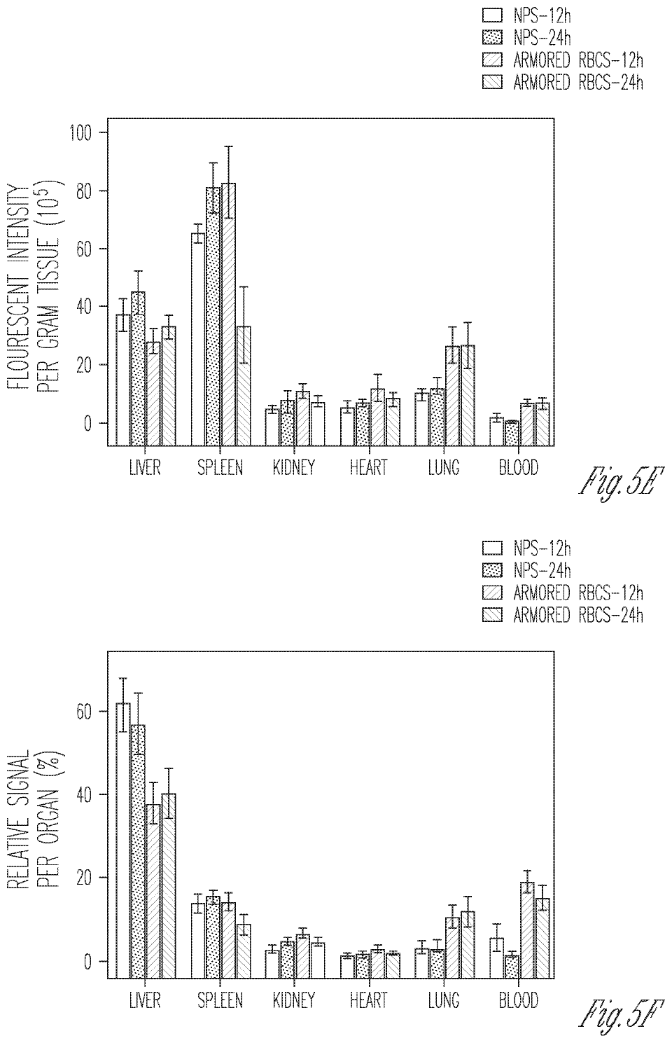

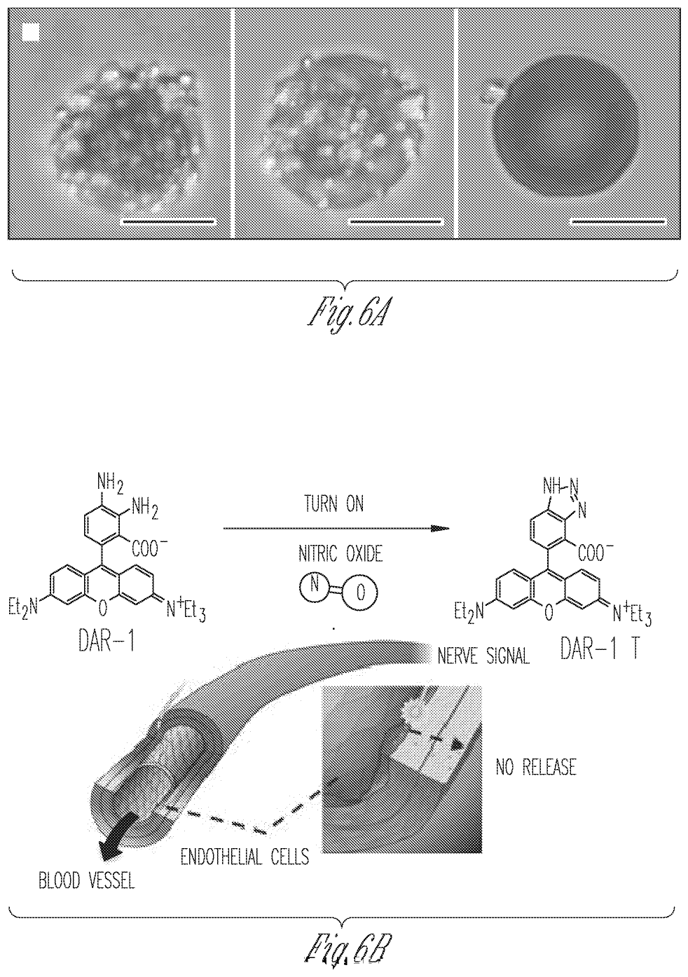

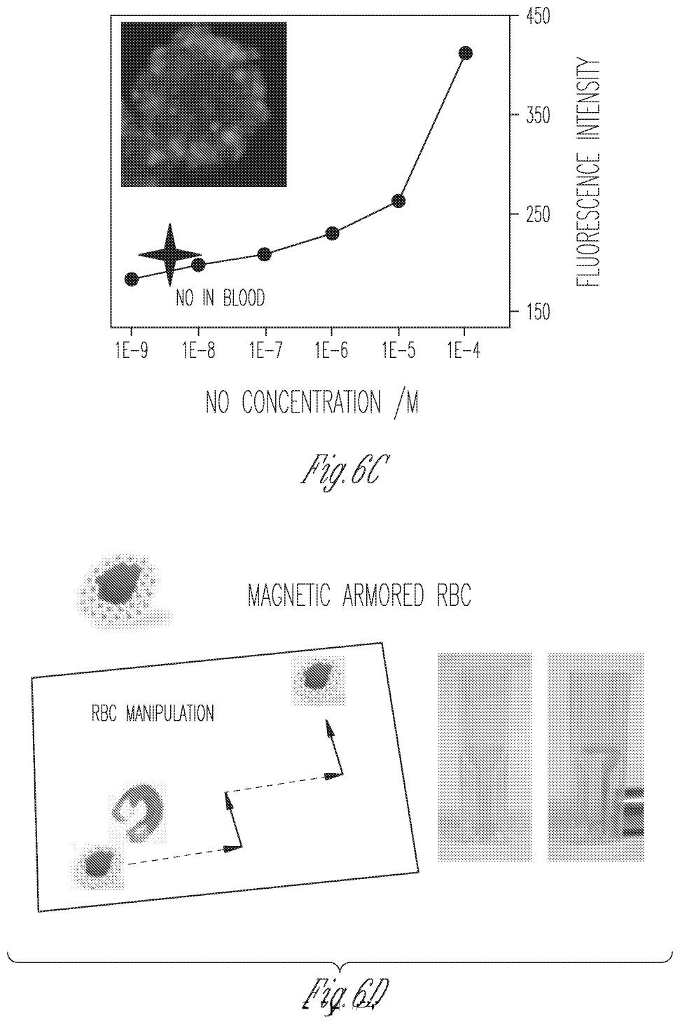

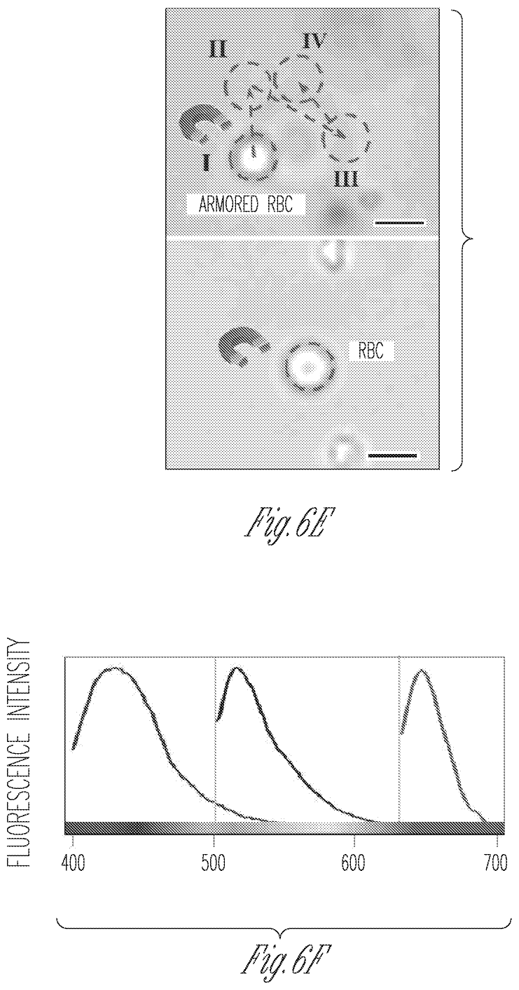

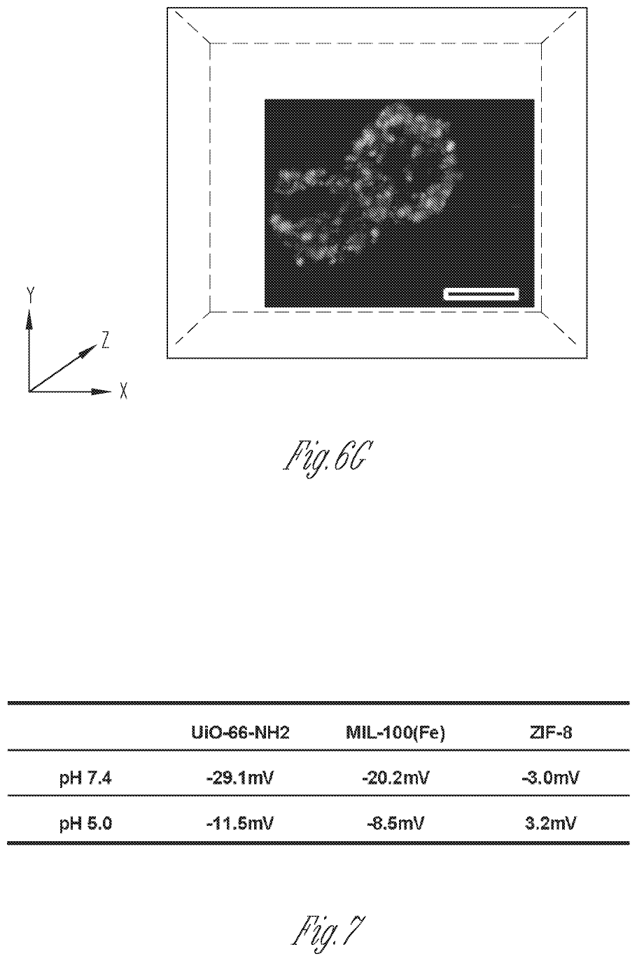

Armored RBCs hold great promise for drug delivery applications. In order to function as a Trojan horse, the encapsulation shell of Armored RBCs plays a pivotal role. Not only its synthesis, but also its triggered disassembly enables Armored RBCs to serve as a nanoparticle reservoir and source fulfilling the Trojan Horse concept. The designed armored RBCs, based on UiO-66-NH₂ MOF NPs exhibit this behavior. Their shell can be disassembled in ethylenediamine-tetraacetic acid (EDTA) solution in a programmed fashion due to the responsive nature of metal-phenolic complexation. The armored MOF shell can be progressively disassembled over the time course of 15 min (as shown by fluorescence microscopy for UiO-66-NH₂-covered RBCs labeled with fluorescein isothiocyanate (FIG. 6a)). Note that the EDTA etching solution (20 mM, pH 7.4) has a negligible impact on RBCs. Upon EDTA-induced detachment of MOF NPs for 15 min, the armored MOF shells were almost completely removed and no defects were observed on the RBC surface suggesting that the RBCs can reversibly return to their original morphology. This on-demand protection shell formation and degradation capability provides RBCs a novel capability reminiscent of the germination of natural spores.

To demonstrate the sensor capabilities of Armored RBCs, the modular properties of MOF NPs were used to design Armored RBCs that detect nitric oxide (NO) in blood. NO is a key signaling molecule acting as a potent vasodilator that relaxes the arteries (FIG. 6b) (Jiang et al., 2013). Hybrid MSN@ZIF-8 NPs (FIG. 14) were used as exoskeleton building blocks wherein 4,5-Diamino-rhodamine B (DAR-1), serving as a fluorescent probe, was pre-loaded within the MSN mesopores. NO-triggered fluorescence was observed for the DAR-1 probe, after its ring-closure and transition from the weakly fluorescent diamino structure to the strongly fluorescent triazole state (Kojima et al., 2001). The sensitivity of designed DAR-1-loaded Armored RBCs was determined by exposing the encapsulated cells to freshly prepared NO solutions with different NO concentrations. The fluorescence intensity increases monotonically with increasing NO concentration (FIG. 6c), which we determined as integrated fluorescence over the sample cuvette via fluorescence wide field imaging of DAR-1-loaded Armored RBCs (see for example, FIG. 6c (Inset) at 100 μM NO and 5 min incubation time). By incubating the sensing Armored RBCs in fresh blood for 5 min, the NO concentration in fresh blood could be determined to be 8.6 nM, which is in line with literature values (Liu et al., 2007). This study demonstrates the potential of our designed Armored RBCs as sensors not only for real-time monitoring and detection of NO in blood, but also further measurements like cellular pH or ROS and redox potentials depending on chosen fluorophores (Lou et al., 2015).

Armed RBCs inherit the collective properties of their MOF or other NP building blocks imparting non-native desirable properties. To demonstrate this idea, magnetic Armored RBCs were created that can be externally controlled. Based on metal-phenolic linker chemistry, magnetic Fe₃O₄ (˜8.0 nm) embedded in ZIF-8 MOFs (FIG. 17) were super-assembled onto the RBC surface to form a magnetic armored shell. In contrast to native RBCs, the magnetic armored RBCs can now be manipulated via an external magnetic field (FIG. 6d-e). This property is not only of interest for 3-dimensional cell patterning and micro-motorized cellular constructs (Wu et al., 2014; Wang & Pumera, 2015), but also magnetic targeting and controlled delivery of therapeutic agents (Dames et al., 2007). Moreover, the controllable magnetic switching makes multifunctional Armored RBCs clearly a potential candidate for hyperthermia induction and a contrast reagent for clinical tumor and leukemia detection via medical Mill imaging (Perez et al., 2002).

Finally, modular super-assembly of Armored RBCs provides far-reaching possibilities for extensions via co-assembly of different functional MOF- (or NP-) based nano-objects into multimodal nano-structures. These could integrate various, simultaneous functionalities, such as contrast for different imaging modalities, thermal therapies and drug delivery. To introduce Armored RBCs as multimodal super-architecture, multi-fluorescent RBCs were created by incubating native RBCs simultaneously with almost equal concentrations of three different fluorescently labelled MSN@ ZIF-8 NPs in a one-pot process for less than 2 minutes (FIG. 6f-g). The fluorescence spectra of resulting Armored RBCs featured three distinct emission peaks at 428, 515 and 648 nm (FIG. 6f). Confocal fluorescence microscopy images in 3D demonstrated the formation of a continuous exoskeleton and a homogeneous distribution of NPs that preserved the stoichiometry of the synthesis solution (FIG. 6g). This example suggests that the co-assembled Armored MOF shell could introduce a nearly infinite number of functionalities to Armored RBCs and might provide efficient coupling effects between the functional MOF NPs.

### CONCLUSIONS

A general, simple, and modular approach is described for a class of hybrid biomaterials termed “armored cells” with diverse possible functionalities. Using metal-phenolic chemistry native RBCs were encapsulated with MOF NP-based exoskeletons in seconds without RBC lysis. The modularity and simplicity of this method arises from fast MOF NPs super-assembly at the RBC membrane surface and enables the transformation of different MOF building blocks and RBC vehicles into diverse functional hierarchitectures. Armored RBCs preserve the original properties of native RBCs, show enhanced resistances against external stressors, and exhibit extraordinary new properties that are foreign to native RBCs based on the highly modular nature of MOF nanobuilding blocks integrated into the RBC exoskeletons. The presented approach profits from the wide range of variable MOF NPs and opens the door to design of multimodal nano-superstructures for multimodal imaging, image-guided therapies and theranostics. The strategy of Armored RBCs, however, is not restricted to red blood cells alone, but can be further extended to any other cell types like leucocytes playing a pivotal role in immunity and inflammatory processes. We believe our findings will open new avenues for artificially designed cell-inspired functional materials for wide ranging biomedical applications.

### Exemplary Embodiments

A modified viable (functional) vertebrate cell encased in (comprising a coat of) reversibly interlinked metal-organic framework (MOF) nanoparticles is provided. In one embodiment, the cell is a mammalian cell. In one embodiment, the cell is a human cell. In one embodiment, the cell is a red blood cell. In one embodiment, the cell is a hematopoietic cell. In one embodiment, the cell is a B cell, T cell or macrophage cell. In one embodiment, the MOF nanoparticles comprise ZIF-8, MIL-100(Fe), UiO-66, UiO-66-NH₂, magnetic iron oxide (Fe₃O₄) NPs@ZIF-8, or mesoporous silica NP@ZIF-8, or a combination thereof. In one embodiment, the nanoparticles have a diameter of about 100 nm to about 500 nm. In one embodiment, the nanoparticles have a diameter of about 100 nm to about 200 nm. In one embodiment, the nanoparticles have a diameter of about 400 nm to about 500 nm. In one embodiment, the nanoparticles have a diameter of about 5 nm to about 500 nm, about 10 nm to about 300 nm or about 15 nm to about 250 nm. In one embodiment, the nanoparticles comprise Zn, Fe, Zr, or Co. In one embodiment, the nanoparticles comprise iron oxide. In one embodiment, the nanoparticles comprise Au, Ni, Mn, Ti, W, Mg, Al, Cu or Cr. In one embodiment, the nanoparticles are linked via a metal-phenolic interaction. In one embodiment, the nanoparticles are linked via a boronic acid-phenolic acid interaction. In one embodiment, the nanoparticles comprise silica. In one embodiment, the nanoparticles further comprise covalently attached moieties. In one embodiment, the mesopores in the nanoparticles further comprise a moiety. In one embodiment, the moiety comprises an optically detectable molecule, a diagnostic molecule or a therapeutic molecule. In one embodiment, the diagnostic molecule comprises a contrast agent or magnetic molecule. Also provided is a composition comprising a population of the encased cells. In one embodiment, the composition further comprises a buffer, e.g., the buffer has a pH less than 7.4.

In one embodiment, a method of making vertebrate cells encased in reversibly interlinked metal-organic framework (MOF) nanoparticles is provided. The method includes combining a population of vertebrate cells and a population of MOF nanoparticles under conditions that allow for attachment of the MOF nanoparticles to the surface of the cells, thereby providing a mixture; and adding an amount of a ligand to the mixture under conditions that allow for interconnecting the MOF nanoparticles.

In one embodiment, a method of making vertebrate cells encased in reversibly interlinked metal-organic framework nanoparticles is provided. The method includes providing a population of vertebrate cells, a population of MOF nanoparticles and a ligand; and adding the MOF nanoparticles and ligand to the cells under conditions that allow for interconnecting the MOF nanoparticles.

In one embodiment, the MOF nanoparticles have a negative charge ranging from −3.0 to −30 mV in 0.2× PBS. In one embodiment, the ligand comprises tannic acid, epigallocatechin gallate, epicatechin gallate myricetin, quercetin, quercetagetin, eupafolin, luteolin, scutellarein, dicaffeoylquinic, theaflavin, heaflavin-3′-gallate (TF1). theaflavin-3, 3′-digallate (TF2) or a combination thereof. In one embodiment, the MOF nanoparticles are in an acidic isotonic buffer that alters the zeta potential of the MOF nanoparticles, e.g., the buffer ranges from pH 5 to pH 7. In one embodiment, the cells are red blood cells. In one embodiment, the MOF nanoparticles comprise ZIF-8, MIL-100(Fe), UiO-66, UiO-66-NH₂, magnetic iron oxide (Fe₃O₄) NPs@ZIF-8, or mesoporous silica NP@ZIF-8, or a combination thereof. In one embodiment, the nanoparticles have a diameter of about 100 nm to about 500 nm. In one embodiment, the nanoparticles have a diameter of about 100 nm to about 200 nm. In one embodiment, the nanoparticles have a diameter of about 400 nm to about 500 nm. In one embodiment, the nanoparticles further comprise a covalently attached moiety, e.g., the moiety is a cell targeting moiety, for instance, an antibody or an antigen binding fragment thereof. In one embodiment, mesopores in the nanoparticles further comprise a molecule. In one embodiment, the moiety or molecule comprises an optically detectable molecule, a diagnostic molecule or a therapeutic molecule. In one embodiment, the diagnostic molecule comprises a contrast agent or a magnetic molecule.

In one embodiment, a method to disrupt linkages in vertebrate cells encased in reversibly interlinked metal-organic framework (MOF) nanoparticles, which includes contacting a population of vertebrate cells encased in reversibly \interlinked metal-organic framework nanoparticles with an agent that disrupts the linkages, e.g., a metal chelator.

### REFERENCES

Anselmo, A. C. et al., ACS Nano 7, 11129-11137 (2013).

Anselmo, A. C. et al., Biomaterials 68, 1-8 (2015).

Avci, C. et al., Nat. Chem. 10, 78-84 (2018).

Bai, G.; Song, Z.; Geng, H.; Gao, D.; Liu, K.; Wu, S.; Rao, W.; Guo, L. & Wang, J. Adv. Mater. 29, 1606843 (2017). DOI: 10.1002/adma.201606843

Biggs, C. I. et al., Nat. Commun., 8, 1546 (2017).

Bobbitt, N. S. et al., Chem. Soc. Rev., 46, 3357 (2017).

Bondar, O. V., Saifullina, D. V., Shakhmaeva, I. I., Mavlyutova, I. I & Abdullin, T. I. Acta Naturae. 4, 78-81 (2012).

Brenner, J. S. et al., Nat. Commun. 9, 2684 (2018).

Cavka, J. H. et al., J. Am. Chem. Soc. 130, 13850-13851 (2008).

Chao & Liu, Bioconjugate Chem. 29, 852-860 (2018).

Dames, P. et al., Nat. Nanotech. 2, 495-499 (2007).

Denny Jr., M. S., Moreton, J. C., Benz, L. & Cohen, S. M. Nat. Rev. Mater. 1, 16078 (2016)

Doshi, N.; Zahr, A. S.; Bhaskar, S.; Lahann, J. & Mitragotri, S. Proceedings of the National Academy of Sciences 106, 21495-21499 (2009). DOI: org/10.1073/pnas.0907127106

Durfee, P. N.; Lin, Y; Dunphy, D. R.; Muniz, A. J.; Bulter, K. S.; Humphrey, K. R.; Lokker, A. J.; Agola, J. O.; Chou, S. S.; Chen, I.; Wharton, W.; Townson, J. L.; Willman, C. L. & Brinker, C. J. ACS Nano 10, 8325-8345 (2016). DOI: 10.1021/acsnano.6b02819

Ejima, H. et al., Science 341, 154-157 (2013).

Freund, R., Lächelt, U., Gruber, T., Rühle, B. & Wuttke, S. ACS Nano, 12, 2094-2105 (2018).

Furukawa, E. H., Cordova, K. E., O'Keeffe, M. & Yaghi, O. M. Science 341, 1230444 (2013).

Godfrin, Y. et al., Expert Opin Bio Ther. 12, 127-133 (2012).

Guo, J. et al., Nat. Nanotech. 11, 1105-1111 (2016).

Hendon, C. H., Rieth, A. J., Korzyński, M. D. & Dincă, M. ACS Cent. Sci. 3, 554-563 (2017).

Horcajada, P. et al., Chem. Rev. 112, 1232-1268 (2012).

Horcajada, P. et al., Chem. Commun. 2820-2822 (2007).

Hu, J.; Zhang, L.; Aryal, S.; Cheung, C.; Fang, R. H. & Zhang, L. PNAS 108,10980-10985 (2011). DOI:10.1073/pnas.1106634108

Jiang, S. et al., Nat. Commun. 4, 2225 (2013).

Kalmutzki, M. J., Diercks, C. S. & Yaghi, O. M. Adv. Mater. 30, 1704304 (2018).

Kim, H. et al., Science 356, 430-434 (2017).

Kim, J. Y. et al., Chem. Asian J 11, 3183-3187 (2016).

Kojima, H. et al., Anal. Chem. 73, 1967-1973 (2001).

Li, Q.; Barrett, D. G.; Messersmith, P. B. & Holten-Anderson, N. ACS Nano 10, 1317-1324 (2016). DOI: 10.1021/acsnano.5b06692

Lismont, M., Dreesen, L. & Wuttke, S. Adv. Funct. Mater. 27, 1606314 (2017).

Liu, X., Yan, Q., Baskerville, K. L. & Zweier, J. L. J. Biol. Chem. 282, 8831-8836 (2007).

Lou, Z., Li, P. & Han, K. Acc. Chem. Res. 48, 1358-1368 (2015).

Lu, G.; Cui, C.; Zhang, W.; Liu, Y. & Huo, F. Chem. Asian J. 8, 69-72 (2013). DOI: 10.1002/asia.201200754

Magnani, M. & Rossi, L. Expert Opin. Drug. Deliv. 11, 677-687 (2014).

Mansouri, S., Merhi, Y., Winnik, F. M. & Tabrizian, M. Biomacromolecules 12, 585-592 (2011).

McDonald, T. M. et al., Nature 519, 303-308 (2015).

Mukthavaram, R., Shi, G., Kersari, S. & Simberg, D. J Control Release 183, 146-153 (2014).

Pan, D. C. et al., Sci. Rep. 8, 1615 (2018).

Park, J. H. et al., Adv. Mater., 26, 2001-2010 (2014).

Park, J. H., Hong, D., Lee, J. & Choi, I. S. Acc. Chem. Res. 49, 792-800 (2016).

Park, T. et al., Polymer 9, 140 (2017).

Perez, J. M., Josephson, L., O'Loughlin, T., Högemann, D. & Weissleder, R. Nat. Biotech. 20, 816-820 (2002).

Pierigè, F., Serafini, S., Rossi, L. & Magnani, M. Adv. Drug Deliv. Rev. 60, 286-295 (2008).

Rahim, Md. A., Kristufek, S. L., Pan, S., Richardson, J. J. & Caruso, F. Angew. Chem. Int. Ed. 57, 2-26 (2018).

Shi, et al. Proc. Natl. Acad. Sci. 111, 10131-10136 (2014).

Stassen, I. et al., Chem. Soc. Rev., 46, 3185-3241 (2017).

Villa, C. H. et al., Ther. Deliv. 6, 795-826 (2015).

Villa, C. H., Anselmo, A. C., Mitragotri, S. & Muzykantov, V. Adv. Drug Deliv. Rev. 106, 88-103 (2016).

Wang et al., Chem. Sci., 5, 3463-3468 (2014).

Wang, C. et al., Adv. Mater. 26, 4794-4802 (2014).

Wang, H. & Pumera, M. Chem. Rev. 115, 8704-8735 (2015).

Wu, Z. et al., ACS Nano 8, 12041-12048 (2014).

Wuttke, S.; Braig, S.; Preiβ, T.; Zimpel, A.; Sicklinger, J.; Bellomo, C.; Rädler, J.; Vollmar, A. & Bein, T. Chem. Commun. 51, 15752-15755 (2015). DOI: 10.1039/c5cc06767g.

Yan, J., Yu, J., Wang, C. & Hu. Z. Small Methods 1, 1700270 (2017).

Yoo, J.-W., Irvine, D. J., Discher, D. E. & Mitragotri, S. Nat. Rev. Drug Discovery 10, 521-535 (2011).

Zhu, W. et al., Adv. Funct. Mater. 28, 1705274 (2018).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A modified vertebrate cell comprising a vertebrate cell encased in reversibly interlinked metal-organic framework (MOF) nanoparticles.

2. The modified cell of claim 1 which is a mammalian cell.

3. The modified cell of claim 2 which is a human cell.

4. The modified cell of claim 1 which is a non-adherent cell.

5. The modified cell of claim 1 which is a red blood cell.

6. The modified cell of claim 1 which is a hematopoietic cell.

7. (canceled)

8. The modified cell of claim 1 wherein the MOF nanoparticles comprise ZIF-8, MIL-100(Fe), UiO-66-NH₂, magnetic iron oxide (Fe₃O₄) NPs@ZIF-8, or mesoporous silica NP@ZIF-8, or a combination thereof.

9. The modified cell of claim 1 wherein the nanoparticles have a diameter of about 100 am to about 500 nm, about 100 nm to about 200 nm or about 400 nm to about 500 nm.

10-11. (canceled)

12. The modified cell of claim 1 wherein the nanoparticles further comprise a covalently attached moiety which optionally is a cell targeting moiety, an optically detectable molecule, a diagnostic molecule or a therapeutic molecule.

13. (canceled)

14. The modified cell of claim 1 wherein the mesopores in the nanoparticles further comprise a molecule which is optionally an optically detectable molecule, a diagnostic molecule or a therapeutic molecule.

15-20. (canceled)

21. A method of making vertebrate cells encased in reversibly interlinked metal-organic framework nanoparticles, comprising:
providing a population of vertebrate cells, a population of MOF nanoparticles and a ligand; and
adding the MOF nanoparticles and ligand to the cells under conditions that allow for interconnecting the MOF nanoparticles via the ligand.

22. The method of claim 21 wherein the MOF nanoparticles have a negative charge ranging from −3.0 to −30 mV in 0.2× PBS.

23. The method of claim 21 wherein the ligand comprises tannic acid, epigallocatechin gallate, epicatechin gallate myricetin, quercetin, quercetagetin, eupafolin, luteolin, scutellarein, dicaffeoylquinic, theaflavin, heaflavin-3′-gallate (TF1), theaflavin-3, 3′-digallate (TF2) or a combination thereof.

24. The method of claim 21 wherein the MOF nanoparticles are in an acidic isotonic buffer that alters the zeta potential of the MOF nanoparticles.

25. The method of claim 24 wherein the buffer ranges from pH 5 to pH 7.

26. The method of claim 21 wherein the cells are red blood cells.

27. The method of claim 21 wherein the MOF nanoparticles comprise ZIF-8, MIL-100(Fe), UiO-66, UiO-66-NH₂, magnetic iron oxide (Fe₃O₄) NPs@ZIF-8, or mesoporous silica NP@ZIF-8, or a combination thereof.

28-30. (canceled)

31. The method of claim 21 wherein the nanoparticles further comprise a covalently attached moiety.

32. (canceled)

33. The method of claim 21 wherein the mesopores in the nanoparticles further comprise a molecule.

34-35. (canceled)

36. A method to disrupt linkages in vertebrate cells encased in reversibly interlinked metal-organic framework (MOF) nanoparticles, comprising:
contacting a population of vertebrate cells encased in reversibly interlinked metal-organic framework nanoparticles with an agent that disrupts the linkages.

37. (canceled)
